(19) United States
(12) Patent Application Publication
(10) Pub. No.: US 2013/0052170 A1
(43) Pub. Date: Feb. 28, 2013

(54) GRAFTING MATERIAL FOR GENETIC AND CELL THERAPY

(71) Applicants: Osam Mazda, Kyoto-shi (JP); Tsunao Kishida, Kyoto-shi (JP)

(72) Inventors: Osam Mazda, Kyoto-shi (JP); Tsunao Kishida, Kyoto-shi (JP)

(73) Assignee: KYOTO PREFECTURAL PUBLIC UNIVERSITY CORPORATION, Kyoto-shi, Kyoto (JP)

(21) Appl. No.: 13/695,603

(22) PCT Filed: Apr. 28, 2011
(86) PCT No.: PCT/JP2011/060488

§ 371 Date: Oct. 31, 2012

(30) Foreign Application Priority Data
Apr. 30, 2010 (JP) 2010-104754

(51) Int. Cl. A61K 48/00; C12N 5/10; A61P 3/10; A61P 3/04; A61P 3/00; A61P 29/00; A61P 1/00; A61P 9/00; A61P 7/04; A61P 3/06; A61P 9/12; A61P 9/04; A61P 35/00; A61P 35/04; A61P 19/10; A61P 1/16; A61P 37/08; A61P 25/00; A61P 17/06; A61P 37/06; A61P 1/18; A61P 9/10; C12N 15/85

(52) U.S. Cl. 424/93.21; 435/455; 435/325

(57) ABSTRACT

Disclosed is a method for producing a grafting material that comprises a step in which a grafting material that expresses a secreted protein is obtained by differentiating iPS cells that have had a gene for a secreted protein introduced therein.

### TECHNICAL FIELD

The present invention relates to a method for treating or preventing diseases that are caused by a deficiency, shortage, or hypofunction of secreted protein, such as malignant tumors, allergic diseases, autoimmune diseases, inflammatory diseases, or hereditary diseases. More specifically, the present invention relates to a grafting material for use in treating these diseases, a method for producing the same, and a method for treating these diseases.

### BACKGROUND ART

Various types of gene therapy have been proposed, wherein genes that are useful for treating malignant tumors, allergic diseases, autoimmune diseases, inflammatory diseases, hereditary diseases, and the like are expressed in a patient's body to attain a therapeutic effect. Procedures to transfer genes that encode soluble proteins, such as cytokines, can be roughly divided into two groups; one is an in vivo method wherein these genes are directly introduced into the patient's body, and the other is an ex vivo method wherein after introducing these genes into some kind of cells, the cell is transplanted to the patient. One example of the in vivo method is that an interferon gene-encoding retroviral vector is injected into the tumor of cancer patients to express the interferon in the tumor cells or the cells near the tumor to achieve a tumor inhibitory effect (Non-patent Literature 1). However, it has been very difficult for an in vivo method to introduce a therapeutic gene to target cells with sufficient efficiency, and to express its gene product over the necessary period at the required amount. Furthermore, it has been almost impossible to remove the introduced gene when side effects are observed or continuation of the treatment becomes unnecessary.

In an ex vivo method, allogeneic or patient-derived cells are transplanted to a patient after introducing a therapeutic gene into the cells. For example, a strategy of introducing an IL-12 gene to autologous fibroblasts, and then transplanting the autologous fibroblasts to the cancer patient has been conducted as a preclinical study (Non-patent Literature 2). Another strategy is reported wherein, after introducing a TNF-alpha gene to allogeneic cells, the allogeneic cells are sealed inside capsules in order to escape from the host's immune rejection, and then transplanted to thecancer patient (Non-patent Literature 3)

However, these transplanted cells do not always survive and continue to express the gene in the body for a long period of time. Depending on the type of cells, the transplanted cells cannot survive in the transplantation site for a long period of time for reasons such as requiring a high amount of oxygen or nutrition. It is not easy for conventional techniques to prepare a sufficient number of cells that are suitable for survivalin a transplantation site for a long period of time. This is because one of the following must be achieved in order to do so, but none of them are easily achieved by conventional techniques. That is, collecting a sufficient number of cells that are suitable for transplantation; collecting a small number of cell strains that are suitable for transplantation and proliferating the cell strains to a sufficient number for transplantation; collecting cells that are easier for collection, proliferating the cells to a sufficient number for transplantation, and differentiating the cells to a cell strain that is suitable for transplantation. It is even more difficult to further introduce the therapeutic gene and keep producing a necessary amount thereof for the required period of time. In contrast, when limited to malignant tumors, a so-called tumor vaccine therapy is performed wherein autologous tumor cells are surgically extracted, then cultured while introducing a therapeutic gene (such as GM-CSF) thereinto, and the result is administered to a patient. The tumor vaccine is usually employed with the expectation that tumor antigens will be presented rather than with the expectation that the product of the introduced secretor gene to work in vivo. In either case, the tumor cells extracted from a patient do not always proliferate in vitro, and tumor vaccine therapy does not always assure that the gene can be effectively introduced, nor that the introduced genes will always be expressed in a necessary amount for the required period of time. Furthermore, the introduced genes cannot always survive for a long period of time after being transplanted into the patient's body. In actuality, such a method does not necessarily achieve favorable treatment results.

Accordingly, if a material obtained by collecting (hopefully by a method that is minimally invasive) the minimum number of patient-derived cells or allogeneic cells (preferably, cells whose HLA is at least partially matched), proliferating the cells to a necessary number, applying a certain treatment such as gene introduction to the cells, and differentiating the cells into cell strains that are able to survive when transplanted into the body (e.g., chondrocytes) can be used as a grafting material, it will be extremely useful in treating malignant tumors, allergic diseases, an autoimmune diseases, inflammatory diseases, hereditary diseases, and the like by an ex vivo method. However, this has not been easy by conventional techniques.

Patent Literature 1 discloses a technique wherein chondrocytes are collected from a joint or the like and cultured, and a gene for a secreted protein for the treatment is introduced.

However, culturing chondrocytes that were collected from a living body is not easy and entails difficulties in the proliferation thereof. Gene introduction is also not sufficiently efficient, causing extreme difficulty in amply expressing the introduced genes.

When the technique of Patent Literature 1 is employed, repeated treatment is practically impossible without repeatedly collecting cartilage from the same patient and repeatedly introducing the genes. This places a considerable burden on the patient and is highly invasive.

### CITATION LIST

### Patent Literature

PTL 1: US2009/0155229

### Non-patent Literature

NPL 1: Yoshida J, et al., Hum Gene Ther. 2004 January; 15(1): 77-86
NPL 2: Cancer Gene Ther. 2009; 16(4): 329-37
NPL 3: Exp. Oncol. 2005; 27(1): 56-60

### SUMMARY OF INVENTION

### Technical Problem

An object of the present invention is to provide an agent for treating diseases caused by the deficiency, shortage, or hypofunction of secreted protein, a therapeutic method thereof, a grafting material effective for treating the diseases, and a production method thereof. Furthermore, even if a disease is not caused by the deficiency, shortage, or hypofunction of secreted protein, if the administration of a certain secreted protein is considered to render an advantageous result in the treatment of the disease, the invention aims to provide an agent for treating such a disease, a method for treating the disease, a grafting material useful for treating the disease, and a production method thereof.

### Solution to Problem

The present invention provides a grafting material comprising transgenic cells for use in an ex vivo method, a method for the preparation thereof, a method for treating diseases using the same, and a bank.

Item 1. A method for producing a grafting material comprising:

differentiating iPS cells introduced with a secreted protein gene and thereby obtaining a grafting material expressing the secreted protein.

Item 2. The method according to Item 1, wherein the secreted protein gene is introduced before, at the same time, or after introducing an iPS inducing factor into cells, preferably during differentiating the iPS cells.

Item 3. The method according to Item 1 or 2, wherein the secreted protein gene is introduced using a viral vector.

Item 4. The method according to Item 3, wherein the viral vector is a retroviral vector.

Item 5. The method according to any one of Items 1 to 4, wherein the grafting material contains chondrocytes.

Item 6. The method according to any one of Items 1 to 5, which further comprises selecting the cell into which the secreted protein gene was introduced.

Item 7. The method according to any one of Items 1 to 6, which further comprises exposing the grafting material to radiation and thereby eliminating the cell proliferation capability.

Item 8. The method according to Item 7, wherein the dosage of the radiation is 15 to 80 Gy, preferably 20 to 40 Gy, and particularly preferably 30 to 40 Gy.

Item 9. The method according to any one of items 1 to 8, wherein the cells obtained by differentiating iPS cells form a cell population or cell mass, which can be transplanted or extracted as one cell population or cell mass.

Item 10. The method according to any one of Items 1 to 9, wherein the grafting material contains somatic cells (dedifferentiated cells) obtained by dedifferentiating somatic cells, inducing differentiation to other somatic cells after or during the dedifferentiation, and introducing the gene into the somatic cells thereduring.

Item 11. A grafting material comprising iPS cell-derived differentiated cells, the grafting material containing a secreted protein gene in such a manner that the secreted protein gene can be expressed.

Item 12. The grafting material according to Item 11, which comprises an iPS inducing factor in the differentiated cells.

Item 13. The grafting material according to Item 12, wherein the iPS inducing factor comprises at least one member selected from the group consisting of the Oct gene family, Klf gene family, Sox gene family, Myc gene family and expression products thereof, and optionally further comprises at least one member selected from the group consisting of Nanog gene family, Lin-28 gene family and expression products thereof.

Item 14. The grafting material according to any one of Items 11 to 13, wherein the differentiated cell is a chondrocyte.

Item 15. The grafting material according to any one of Items 11 to 14, wherein the grafting material is a population or mass of the differentiated cells.

Item 16. The grafting material according to any one of Items 11 to 15, wherein the grafting material contains somatic cells (dedifferentiated cells) obtained by dedifferentiating somatic cells, inducing differentiation to other somatic cells after or during the dedifferentiation, and introducing the gene into the somatic cells thereduring.

Item 17. An agent for treating a disease caused by a deficiency, shortage, or hypofunction of a secreted protein, the agent comprising the grafting material obtained by any one of the methods of Items 1 to 10 or any one of the grafting materials of Items 11 to 16 as an active ingredient.

Item 18. The agent according to Item 17, wherein the secreted protein is at least one member selected from the group consisting of insulin, GLP-1, GLP-1 (7-37) and like GLP-1 receptor agonist polypeptides, GLP-2, interleukins 1 to 33 (e.g., IL-1, IL-2, IL-3, IL-4, IL-5, IL-6, IL-7, IL-8, IL-9, IL-10, IL-11, IL-12, IL-13, IL-15, IL-17, IL-18, IL-21, IL-22, IL-27, IL-28, IL-33), interferons (α, β, γ), GM-CSF, G-CSF, M-CSF, SCF, FAS ligand, TRAIL, leptin, adiponectin, blood coagulation factor XIII/blood coagulation factor IX, lipoprotein lipase (LPL), lecithin cholesterol acyltransferase (LCAT), erythropoietin, apolipoprotein A-I, albumins, atrial natriuretic peptide (ANP), luteinizing hormone-releasing hormones (LHRH), angiostatin/endostatin, endogenous opioid peptides (enkephalins, endorphins and the like), calcitonin/bone morphogenetic proteins (BMP), pancreatic secretory trypsin inhibitors, catalase, superoxide dismutases, and antibodies.

Item 19. The agent according to Item 17 or 18, wherein the disease is at least one member selected from the group consisting of diabetes, obesity, eating disorders, inflammatory bowel diseases, gastrointestinal disorders, vascular disorders, hemophilia, lipoprotein-lipase (LPL) deficiency, hypertriglyceridemia, lecithin cholesterol acyltransferase (LCAT) deficiency, hypoglobulia, low HDL cholesterol, hypoproteinemia, hypertension, heart failure, malignant melanoma, renal cancer, breast cancer, prostatic cancer, cancer metastasis, pain, osteoporosis, malignant tumors, hepatitis, allergies, multiple sclerosis, psoriasis, autoimmune diseases, pancreatitis, ischemic heart diseases and like ischemia reperfusion disorders.

Item 20. A method for treating a disease comprising:

administering the agent of Item 17, 18 or 19 to a patient suffering from any of the diseases of Item 19.

Item 21. A bank of a grafting material obtained by any one of the methods of Items 1 to 10 or any one of the grafting materials of Items 11 to 16.

Item 22. The bank according to Item 21, wherein the grafting material is a chondrocyte.

Item 23. The bank according to Item 21 or 22, wherein the protein secreted by the grafting material is a cytokine, chemokine or antibody.

Item 24. The bank according to Items 21 to 23, wherein the cells forming the grafting material substantially do not have a proliferation potential.

### EFFECTS OF THE INVENTION

In the present invention, it was found that iPS cells are remarkably suitable for ex vivo gene introduction. More specifically, (i) because iPS cells can be established from a patient himself or herself, a grafting material for use in treatment can be obtained using patient-derived cells by differentiating the iPS cells into cells suitable for ex vivo treatment (e.g., chondrocytes); (ii) a large number of cells for treatment can be provided by proliferating iPS cells in vitro; (iii) genes can be introduced into cells that are in the process of differentiating into cells suitable for ex vivo treatment (e.g., chondrocytes).

In the present invention, it was also found that genes can be introduced to and produced in iPS-derived cells more effectively than the case where cells suitable for ex vivo treatment (e.g., chondrocytes) are directly collected from a patient and genes are introduced thereto. In addition, it was found that employing the present invention makes it possible to produce a grafting material that has lost its cell proliferation capability while continuing to produce a gene product, by performing irradiation after introducing genes to iPS-derived cells. This is almost unfeasible by any conventional techniques; therefore, this is a major advantage of the present invention.

The grafting material of the present invention is an excellent source for continuously supplying a secreted protein, because it can introduce many secreted protein genes to iPS cell-derived differentiated cells in such a manner that they can be expressed at high levels. The grafting material of the present invention wherein iPS cell-derived differentiated cells are used as the source of supplying secreted protein is also excellent as an agent for treating diseases caused by a deficiency, shortage, or hypofunction of secreted protein. Furthermore, the present invention is also effective as an agent for treating diseases other than those caused by a deficiency, shortage, or hypofunction of secreted protein if the administration of a certain secreted protein is considered to bring beneficial results to the patient.

### BRIEF DESCRIPTION OF DRAWINGS

FIG. 1 is a summary of the experiment in which mouse iPS cells are infected with a retroviral vector during the differentiation of mouse iPS cells into chondrocytes, and in which primary rabbit chondrocytes are infected with the retroviral vector. See Example 1.

FIG. 2 shows the results of the experiment shown in FIG. 1. In FIG. 2, the arrows indicate EGFP expression cells. See Example 2.

FIG. 3A is a diagram showing a summary of the experiment to compare the gene transfection and expression efficiency between the case when human iPS cell-derived chondrocytes are infected with a retrovirus during differentiation and the case when primary human chondrocytes are infected with the retrovirus.

FIG. 3B shows the results of alcian blue staining in the experiment shown in FIG. 3A. The staining was slightly positive on day 20, and strongly positive on day 23. This shows that human iPS cells were differentiated into chondrocytes. See Example 3.

FIG. 4 shows the results of the experiment shown in FIG. 3A. It is clear from the GFP expression that the infection of human iPS-derived chondrocytes with a retrovirus during differentiation results in very high gene transfection and expression efficiency, compared to the case of the infection of primary human chondrocytes with a retrovirus. See Example 4.

FIG. 5 in the same manner as in FIG. 3A, a retroviral vector containing a secreted luciferase gene was used to infect human iPS-derived chondrocytes during differentiation and primary human chondrocytes, and these chondrocytes were compared. It is clear from the luciferase expression that a much higher gene transfection and expression efficiency can be obtained in the former than in the latter. See Example 5.

FIG. 6 is a summary of an experiment in which chondrocytes differentiated from iPS cells are irradiated with soft X-rays, and the influence of the dose on the cell growth is examined. See Example 6.

FIG. 7 is a summary of the experiment in which chondrocytes differentiated from iPS cells are irradiated with soft X-rays, and the influence of the dose on the cell growth is examined. EB indicates embryoid bodies. See FIG. 7.

FIG. 8 is a summary of the experiment to observe the influence of the dose of soft X-rays on the expression of a plasmid vector introduced into chondrocytes differentiated from iPS cells. See Example 8.

FIG. 9 shows the results of an experiment in which chondrocytes differentiated from iPS cells are transfected with a plasmid vector, irradiated with soft-X rays, and further cultured, and the influence of the dose of soft X-rays on the expression of the transgene is observed. See Example 9.

FIG. 10 shows a summary of a transplant experiment. See Example 10.

FIG. 11 shows diagrams of plasmid vectors. See Example 11.

FIG. 12 shows the data obtained by measuring the mRNA expression of aggrecan (index of chondrocytes) in iPS cells and the cells that were cultured as shown in FIG. 1. See Example 12.

FIG. 13 The left image in FIG. 13 shows a differential interference contrast microscope image of the cells cultured from iPS cells as shown in FIG. 1. The right image in FIG. 13 shows a fluorescence microscope image of the same cells on day 1 after the cells were transfected with pmaxGFP. See Example 13.

FIG. 14 shows data obtained by measuring, using ELISA, the IL-12 p70 levels in the mouse serum on day 1 (left) or day 4 (right) after transplantation. See Example 14.

FIG. 15 shows data obtained by measuring, using a Luc assay, the Luc activity in the mouse serum on day 1 (left) or day 4 (right) after transplantation. See Example 15.

FIG. 16 is a summary of an experiment in which cartilage precursor cells differentiated from iPS cells are infected with a mouse IL-12- or GFP-expressing retroviral vector, irradiated with soft X-rays, and transplanted into mice, so as to measure the IL-12 or GFP levels in the serum. See Example 16.

FIG. 17 shows the results obtained by measuring the IL-12 or GFP levels in the serum. See Example 16.

FIG. 18 is a summary of an experiment in which cartilage precursor cells differentiated from iPS cells are infected with a mouse IL-12-expressing retroviral vector, irradiated with soft X-rays, and then transplanted into mice; and 3 days after transplantation, the serum IL-12 levels are measured in a group that underwent excision of transplanted cartilage masses and a group that did not undergo such excision. See Example 17.

FIG. 19 shows the results obtained by measuring the serum IL-12 levels in Example 17. The vertical axis indicates the serum IL-12 levels (pg/mL).

FIG. 20 is a summary of an experiment to measure the cell viability of iPS-derived embryoid bodies when irradiated with 0-40 Gy of soft X-rays. See Example 18.

FIG. 21 shows the results obtained by measuring the cell viability in Example 18.

FIG. 22 is a summary of an experiment to measure the secreted luciferase (MetLuc2) or GFP levels in the serum of SCID mice when iPS-derived cartilage precursor cells irradiated with 20 Gy of soft X-rays or non-irradiated cartilage precursor cells are transplanted into the SCID mice. See Example 19.

FIG. 23 shows the results obtained by measuring the secreted luciferase (MetLuc2) or GFP levels in the serum in Example 19.

FIG. 24 is a summary of an experiment to measure the tumor size (Example 20) and the viability after tumor transplantation in C57BL/6 mice subcutaneously transplanted with a mouse melanoma B16 cell line (5×10⁵ cells). See Examples 20 and 21.

FIG. 25 shows the results obtained by measuring the tumor volume in Example 20. The vertical axis indicates the tumor volume.

FIG. 26 shows the results obtained by measuring the viability after tumor transplantation in Example 21. The vertical axis indicates the viability.

FIG. 27 is a summary of an experiment to transplant mouse iPS cell-derived chondrocytes (5×10⁶) infected with a retroviral vector containing a mouse IL-12 gene or GFP gene, which was prepared using a Platinum Retroviral Expression System. See Example 22.

FIG. 28 is a summary of an experiment in which a CTL assay is performed on a mouse melanoma B16 cell line after transplantation of mouse iPS cell-derived chondrocytes. See Example 22

FIG. 29 is a summary of an experiment in which a CTL assay is performed in Example 22.

FIG. 30 is a summary of an experiment to transplant mouse iPS cell-derived chondrocytes (5×10⁶) infected with a retroviral vector containing a mouse IL-12 gene or GFP gene, which was prepared using a Platinum Retroviral Expression System. See Example 23.

FIG. 31 is a summary of an experiment in which an NK assay is performed on a mouse melanoma B16 cell line after transplantation of mouse iPS cell-derived chondrocytes. See Example 23.

FIG. 32 shows the results of an experiment of an NK assay in Example 23.

FIG. 33 shows a procedure for preparing, using packaging cells, a retrovirus containing a human Sox9 gene, mouse Klf4 gene, mouse cMyc gene, and GFP gene, and infecting fibroblasts with the retrovirus. See Example 24. FIGS. 33 to 42 (Examples 24 to 29) show that dedifferentiated chondrocytes that produce secreted proteins can be obtained by dedifferentiating somatic cells, inducing differentiation into chondrocytes subsequently to or simultaneously with the dedifferentiation (the chondrocytes obtained by this method are referred to as dedifferentiated chondrocytes), and introducing the gene thereduring. iPS cells of the present invention encompass such cells at the stages of dedifferentiation and differentiation of somatic cells into chondrocytes. Specifically, such dedifferentiation and differentiation of somatic cells into chondrocytes are also encompassed in the dedifferentiation of somatic cells into iPS cells and subsequent differentiation into chondrocytes as defined in the present invention. Further, such gene transfection during the dedifferentiation of somatic cells and the differentiation into chondrocytes is also encompassed in the gene transfection during the differentiation of iPS cells into chondrocytes as defined in the present invention.

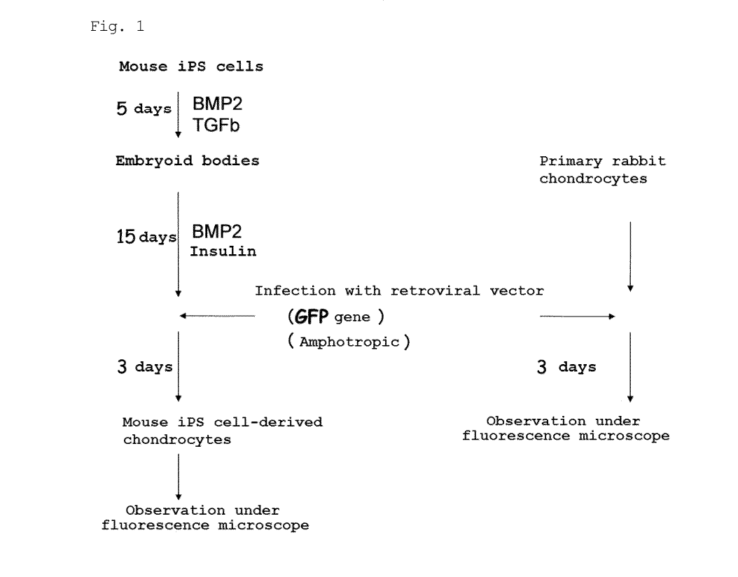

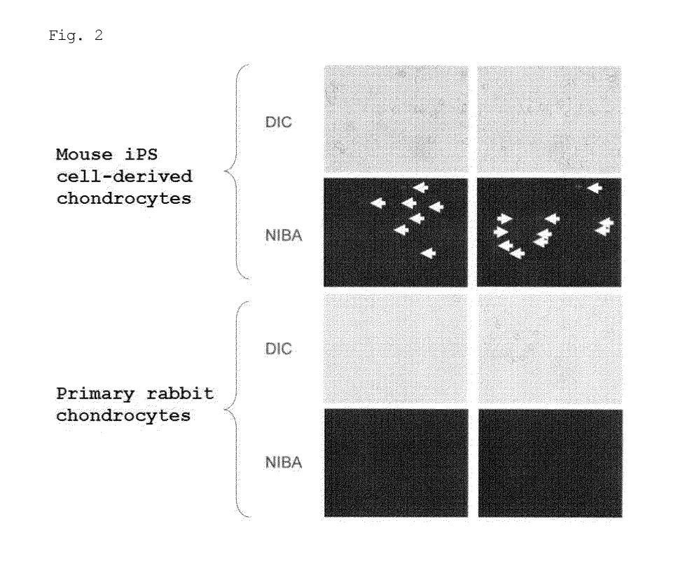

Similarly, dedifferentiated somatic cells obtained by dedifferentiating original somatic cells, inducing differentiation into other somatic cells subsequently to or simultaneously with the dedifferentiation (the somatic cells obtained by this method are referred to as dedifferentiated somatic cells), and introducing the gene thereduring, can also be used for treatment. iPS cells of the present invention encompass such cells at the stage of dedifferentiation and differentiation of original somatic cells into other somatic cells. Therefore, such dedifferentiation and differentiation of original somatic cells into other somatic cells are also encompassed in the dedifferentiation of somatic cells into iPS cells and the subsequent differentiation into somatic cells as defined in the present invention. Further, such gene transfection during the dedifferentiation of original somatic cells and differentiation into other somatic cells is also encompassed in the gene transfection during the differentiation of iPS cells into somatic cells as defined in the present invention.

FIG. 34 shows the results of alcian blue staining performed on day 9 of the infection. See Example 24.

FIG. 35 shows the results of fluorescent observation and alcian blue staining performed on the cells transfected with a GFP gene 2 days after the second infection in Example 25. DIC indicates differential interference, and NIBA indicates a fluorescent image.

FIG. 36 shows a procedure for performing real-time RT-PCR using aggrecan, i.e., a chondrocyte-specific marker gene, and a TaqMan probe and primer set that targets the type II collagen gene. See Example 26.

FIG. 37 shows the results of real time RT-PC in Example 26.

FIG. 38 shows a procedure for measuring mouse IL-12 and luciferase by ELISA in Examples 27 and 28.

FIG. 39 shows the results obtained by measuring mouse IL-12 by ELISA in Example 27.

FIG. 40 shows the luciferase assay results in Example 28.

FIG. 41 shows a procedure for measuring the protein levels of mIL-21 and luciferase in the serum in Example 29.

FIG. 42 shows the results obtained by measuring the protein levels of mIL-21 and luciferase in the serum in Example 29.

FIG. 43 shows the procedure until the measurement of IL-21 in Example 30.

FIG. 44 shows the results obtained by measuring IL-21 in Example 30.

FIG. 45 shows a procedure for producing anti-HA(PR8) antibodies using iPS-derived chondrocytes in Example 31.

FIG. 46 shows the results obtained by measuring the anti-HA(PR8) antibody levels in Example 31.

FIG. 47 shows the results of Example 34.

### DESCRIPTION OF EMBODIMENTS

Unless otherwise indicated, the term “treatment” or “treating” as used herein means any procedure that is applied to a patient while the patient is suffering from a specific disease or disorder and that can reduce the severity of the disease or disorder or one or more symptoms thereof, or retard or slow the progression of the disease or disorder. The term “treatment” as used herein includes “prophylaxis.”

Examples of the target disease to be treated by using the graft material of the present invention include malignant tumors (which include, but are not limited to, melanoma, renal cancer, breast cancer, prostate cancer, and cancer metastasis), pain relief, osteoporosis, hepatitis, allergic diseases, multiple sclerosis, psoriasis, autoirrmune diseases, inflammatory diseases, genetic diseases (which include, but are not limited to, hemophilia A and α2 antitrypsin deficiency), rheumatic diseases, diabetes, obesity, eating disorders, inflammatory bowel diseases, gastrointestinal disorders, vascular disorders, hemophilia, lipoprotein lipase (LPL) deficiency, hypertriglyceridemia, lecithin-cholesterol acyltransferase (LCAT) deficiency, erythrocytopenia, low HDL, hypoproteinemia, hypertension, heart failure, pancreatitis, ischemic heart diseases, and like ischemia reperfusion disorders. In addition to the above diseases, other various diseases that relate to the deficiency, shortage, or hypofunction of secreted proteins are also included within the scope of the target disease. The target disease further includes diseases that are not caused by the deficiency, shortage, or hypofunction of secreted proteins but for which administration of a certain secreted protein is considered to bring beneficial results to the patient.

The present invention can be used for the treatment of diseases, as well as for other purposes, such as health promotion and beauty (for example, when the secreted protein is collagen). Any treatment provided to humans for health promotion and beauty is also called “treatment” for the sake of convenience in this specification. In this case, reference to a “patient” can be deemed to refer to a “healthy person” or a “human,” and reference to “disease” can be deemed to refer to “health promotion,” “beauty,” etc.

The present invention can be used for humans, as well as for animals kept as pets, such as dogs and cats, and animals kept as livestock, such as cows, horses, pigs, sheep, and chickens. In this case, reference to a “patient” can be deemed to refer to an “diseased animal” or “animal.”

The term “graft material” refers to a material introduced into a living body to express a secreted protein encoded by a foreign secreted protein gene in the body, in anticipation of its effect. “Graft material” includes a material that is grafted to the same or different individuals after a secreted protein gene is transferred in vitro.

The term “iPS cells” refers to cells considered to have pluripotency and self-renewal capacity artificially induced by initializing somatic cells. Somatic cells may be derived from an embryo, fetus, or living body, and may be derived from any animal species, such as mice and humans.

Examples of cells into which iPS cells are induced to differentiate include, but are not limited to, fibroblasts, epithelial cells (e.g., skin epidermal cells, corneal epithelial cells, conjunctival epithelial cells, oral mucosal epithelium, follicle epithelial cells, oral mucosal epithelial cells, airway mucosal epithelial cells, and intestinal mucosal epithelial cells), osteocytes, osteoblasts, osteoclasts, mammary gland cells, ligament cells, chondrocytes, vascular endothelial cells, hepatocytes, pancreatic cells, adipocytes, nerve cells, cardiomyocytes, retinal cells, splenic cells, bone marrow cells, mesangial cells, Langerhans cells, epidermal cells, immune cells (e.g., macrophages, T cells, B cells, natural killer cells, mast cells, neutrophils, basophils, eosinophils, monocytes, and leucocytes), megakaryocytes, synoviocytes, stromal cells, and the like. Examples of preferable differentiated cells include chondrocytes, osteocytes, fibroblasts, and the like.

“iPS cells” as used herein include both de-differentiated cells and reprogrammed cells, i.e., cells de-differentiated by an appropriate means, and cells reprogrammed by an appropriate means, such as introducing a specific set of genes. iPS cells do not necessary have pluripotency in a strict sense of the word, but include a wide variety of cells, such as cells de-differentiated into mesenchymal stem cell-like cells from somatic cells, and intermediate cells obtained during the process of inducing original somatic cells (e.g., fibroblasts) into other cells (e.g., chondrocytes) by sequential or simultaneous induction of de-differentiation and differentiation, as shown in Example 24.

iPS inducing factors for initializing differentiated cells are not particularly limited, but preferably include a set of genes or gene-expression products thereof respectively selected from the Oct gene family, Klf gene family, and Sox gene family. In view of the efficiency of establishing iPS cells, a set of genes further including a gene of the myc gene family or an expression product thereof is preferable. Genes that belong to the Oct gene family include, for example, Oct3/4, Oct1A, Oct6, and the like. Genes that belong to the Kif gene family include, for example, Klf1, Klf2, Kif4, Klf5, and the like. Genes that belong to the Sox gene family include, for example, Sox1, Sox2, Sox3, Sox7, Sox15, Sox17, Sox18, and the like. Genes that belong to the myc gene family include c-myc, N-myc, L-myc, and the like. Gene products of the myc gene family may be substituted with a cytokine. Examples of such cytokines include SCF and bFGF. In view of iPS cell production efficiency, the introduction of genes of the above gene families is preferable; however, at least one protein, which is a gene-expression product of one of the genes belonging to the above gene families, may be introduced into differentiated cells to produce iPS cells.

Examples of iPS inducing factors include, in addition to the above-mentioned sets, a set of Nanog gene and lin-28 gene with a gene of the Oct gene family and a gene of the Sox gene family. The above set of genes may be introduced into cells with other gene products, such as immortalization-inducing factors.

Alternatively, iPS inducing factors may consist of expression products of genes each selected from the Oct gene family, Klf gene family, and Sox gene family (e.g., Oct protein, Klf protein, and Sox protein). In view of iPS cell establishment efficiency, a set of proteins further including a protein encoded by the c-myc gene family is more preferable. When such a protein is introduced to produce iPS cells, the possibility of canceration is lowered or eliminated, which is thus preferable. Alternatively, a small molecule may be used instead of such a protein. The use of an episomal vector or a sendaiviral vector to produce iPS cells also lowers the possibility of canceration, which is thus preferable. Alternatively, a combination of such a gene, protein, small molecule, etc., may also be used.

All of the above genes are highly conserved among vertebrates. The term “gene” referred to in this specification includes its homologues unless the name of a particular animal is indicated. “Gene” also includes polymorphisms and mutated genes that have a function comparable to that of wild-type gene products. iPS cells can be produced by known methods, for example, according to “Induction of pluripotent stem cells from adult human fibroblasts by defined factors,” Takahashi K, Tanabe K, Ohnuki M, Narita M, Ichisaka T, Tomoda K, Yamanaka S, Cell, 2007 Nov. 30; 131(5): 861-72, and “Generation of mouse-induced pluripotent stem cells with plasmid vectors,” Okita K, Hong H, Takahashi K, Yamanaka S, Nat. Protoc. 2010; 5(3): 418-28. More specifically, when the iPS inducing factor is a protein that is functional in cells, it is preferable that a gene encoding the protein is introduced into an expression vector, and the expression vector is introduced into target differentiated cells, such as somatic cells, and intracellularly expressed. Although the expression vector to be used is not particularly limited, a viral vector is preferable. In particular, a retroviral vector or a lentiviral vector is preferably used. Alternatively, an iPS inducing factor may be introduced into cells by binding a peptide called a “protein transduction domain (PTD)” to the protein and adding the fusion protein to a culture medium. If some of the iPS inducing factors have been expressed in differentiated cells for use as the starting material for iPS cells, it is not necessary to introduce the proteins externally. Instead of introducing a reprogramming factor or a gene of the reprogramming factor, a small molecule may be used to induce iPS cells. For example, iPS cells can be induced according to the methods described in “Generation of induced pluripotent stem cells using recombinant proteins,” Zhou H, Wu S, Joo J Y, Zhu S, Han D W, Lin T, Trauger S, Bien G, Yao S, Zhu Y, Siuzdak G, Scholer H R, Duan L, Ding S, Cell Stem Cell, 2009 May 8; 4(5): 381-4, and “Generation of human induced pluripotent stem cells by direct delivery of reprogramming proteins,” Kim D, Kim C H, Moon J I, Chung Y G, Chang M Y, Han B S, Ko S, Yang E, Cha K Y, Lanza R, Kim K S, Cell Stem Cell, 2009 Jun. 5; 4(6): 472-6.

The differentiation-inducing medium for differentiating iPS cells is not particularly limited, and may be, for example, the media described in “Endochondral bone tissue engineering using embryonic stem cells,” Jukes J M, Both S K, Leusink A, Sterk L M, van Blitterswijk C A, de Boer J. Proc Natl Acad Sci USA, 2008 May 13; 105(19): 6840-5; “Induction of chondro-, osteo- and adipogenesis in embryonic stem cells by bone morphogenetic protein-2: effect of cofactors on differentiating lineages,” zur Nieden N I, Kempka G, Rancourt D E, Ahr H J, BMC Dev Biol., 2005 Jan. 26; 5:1; and “Embryonic stem cell differentiation models: cardiogenesis, myogenesis, neurogenesis, epithelial and vascular smooth muscle cell differentiation in vitro,” Guan K, Rohwedel J, Wobus A M, Cytotechnology, 1999 July; 30(1-3): 211-26.

The expression of a secreted protein can be easily confirmed by culturing the graft material in a medium and detecting the protein secreted in the medium using an immunoassay, such as ELISA.

The graft material of the present invention may be a cell that can express a secreted protein, but is preferably a cell mass or cell population because this allows all to be removed after introduction into the living body. For example, secretion of a secreted protein used for anti-cancer purposes is preferably halted after shrinking or disappearance of the cancer. In this case, the graft material introduced or embedded into the living body can be partially or completely removed.

The graft material of the present invention may contain an extracellular matrix (ECM). Examples of ECM components include collagen, fibronectin, vitronectin, laminin, heparan sulfate, proteoglycan, glycosaminoglycan, chondroitin sulfate, hyaluronan, dermatan sulfate, keratin sulfate, elastin, and combinations of two or more of the above. Such an ECM component can be used by gelling the ECM component and mixing the gel with differentiated cells that form a graft material. The ECM component and differentiated cells are introduced into a scaffold having a gel or paste network structure, a fibrous structure, a flat (disc) structure, a honeycomb structure, or a sponge-like structure to form a graft material of a three-dimensional structure.

Examples of the secreted protein of the present invention include hormones, cytokines, chemokines, and the like. Specific examples of such secreted proteins include insulin, GLP-1, GLP-1 (7-37), and like GLP-1 receptor agonist polypeptides, GLP-2, interleukins 1 to 33 (e.g., IL-1, IL-2, IL-3, IL-4, IL-5, IL-6, IL-7, IL-8, IL-9, IL-10, IL-11, IL-12, IL-13, IL-17, IL-18, IL-21, IL-22, IL-27, IL-33), interferon (α, β, γ), GM-CSF, G-CSF, M-CSF, SCF, FAS ligands, TRAIL, leptin, adiponectin, blood coagulation factor VIII/blood coagulation factor IX, lipoprotein lipase (LPL), lecithin-cholesterol acyltransferase (LCAT), erythropoietin, apoA-I, albumin, atrial natriuretic peptide (ANP), luteinizing hormone releasing hormone (LHRH), angiostatin/endostatin, endogenous opioid peptides (enkephalins, endorphins, etc.), calcitonin/bone morphogenetic protein (BMP), pancreatic secretory trypsin inhibitors, catalase, superoxide dismutase, anti-TNF-α antibody, soluble IL-6 receptor, IL-1 receptor antagonist, α2 antitrypsin, and like antibodies, and other soluble proteins. A gene encoding such a soluble protein whose expression is therapeutically relevant for treating a certain disease can also be used. Alternatively, a gene encoding a peptide is also usable. In this case, reference to a “soluble protein” can be deemed to refer to a “peptide,” and the present invention can be used for treating a disease for which the peptide is effective.

Examples of the set of secreted protein and disease include, but are not limited to, insulin/diabetes; glucagon-like peptide-1 (GLP-1)/diabetes, obesity, and eating disorders; GLP-2/inflammatory enteropathy and gastrointestinal disorders associated with cancer chemotherapy, etc.; leptin/obesity and lipodystrophic diabetes; adiponectin/diabetes and angiopathy; blood coagulation factors VIII and IX/hemophilia; lipoprotein lipase (LPL)/LPL deficiency and hypertriglyceridemia; lecithin cholesterol acyltransferase (LCAT)/LCAT deficiency; erythropoietin/erythropenia; apoA-I/hypo-HDL cholesterolemia; albumin/hypoproteinemia; atrial natriuretic peptide (ANP)/hypertension and cardiac failure; luteinizing hormone releasing hormone (LHRH)/breast cancer and prostate cancer; angiostatin and endostatin/angiogenesis and metastasis inhibition; morphine receptor agonist peptide (endogenous opioid peptide)/pain relief; calcitonin and bone morphogenetic factor (BMP)/osteoporosis; interferon-α and interferon-β/malignant tumors; interferon-γ/malignant tumors, hepatitis, and allergies; interferon-β1/multiple sclerosis; interleukin-1α or interferon-1β/malignant tumors; interleukin-4/psoriasis; interleukin-10/autoimmune diseases; interleukin-12/malignant tumors; pancreatic secretory trypsin inhibitor/pancreatitis; superoxide dismutase/ischemic heart diseases and angiopathy; tumor necrosis factor-α (TNF-α) solubilized receptor/rheumatoid arthritis; solubilized IgE receptor/allergies; solubilized IgA receptor/food allergies; solubilized cytotoxic T lymphocyte antigen-4 (CTLA4)/autoimmune diseases; solubilized CD40 ligand/immunological disorders; dominant negative blood coagulation factor VIIa/thrombosis; and fibroblast growth factor (FGF) solubilized receptor/vascular intimal thickening.

The secreted protein gene may be introduced into differentiated cells before or at the same time as iPS inducing factors are introduced into the cells. More preferably, after a secreted protein gene is introduced into iPS cells, the cells are induced to differentiate. Still more preferably, after iPS cells are partway differentiated into cells suitable for gene transfer (e.g., embryoid bodies), a secreted protein gene is introduced into the iPS-derived cells, and the resulting cells are further differentiated into cells suitable for transplantation. This is because gene transfer can be efficiently performed during the process of differentiation from iPS cells. Although the secreted protein gene may be introduced by a plasmid, using a viral vector is preferable in view of transfer efficiency and stable maintenance. The phrase “stable maintenance” as used herein means that the secreted protein gene is passed on to daughter cells during cell division. More specifically, this phrase means incorporation of the secreted protein gene into a cell chromosome. The differentiated cell contained in the graft material of the present invention preferably has a foreign secreted protein gene stably introduced by a chromosomal integration viral vector. More preferably, the foreign secreted protein gene is introduced by a retroviral vector.

Preferably, the secreted protein gene is stably introduced by a chromosomal integration viral vector. More preferably, the secreted protein gene is introduced by a retroviral vector. The secreted protein gene in the retrovirus can be transcribed by LTR or may be expressed from another promoter inside the vector. For example, a constitutive expression promoter such as a CMV promoter, EF-1α promoter, or CAG promoter, or a desired inducible promoter may be used. Alternatively, a chimeric promoter, in which a portion of LTR is substituted with another promoter, may be used.

However, if the secreted protein gene is introduced simultaneously with iPS inducing factors into cells by a retroviral vector, the secreted protein gene is integrated into a chromosome but the expression is assumed to be suppressed (silenced), which is thus not preferable. Accordingly, after iPS cells are partway differentiated, the secreted protein gene is introduced. In this case, a graft material capable of expressing the secreted protein gene is efficiently obtained, which is thus preferable.

A retroviral vector is stably integrated into a cell chromosome and has the ability to express a transgene for a long period. However, the transfer efficiency and persistence of the expression of the transgene depends on the cell type. For example, in some cases, the expression of a gene introduced by a retroviral vector persists while the cells are growing, but the gene expression stops when cell growth stops. The suppression of the expression of a secreted protein gene is often observed particularly after the gene is introduced into the body by an in vivo or ex vivo method. However, when the present inventors introduced a secreted protein gene into iPS-derived cells using a retroviral vector, surprisingly, expression of the secreted protein gene persisted extremely stably, both in vitro and in vivo. The expression of the secreted protein gene was stable in undifferentiated iPS cells and in differentiated iPS-derived cells. The expression persisted for 4 days or longer in an in vitro culture. The expression persisted even longer when the iPS-derived cells were transplanted into the body. Accordingly, the graft material of the present invention, which comprises iPS-derived differentiated cells having a secreted protein gene stably introduced therein, can be used as an implant that is a source of a secreted protein and that stably expresses the gene for a long period of time.

In order to prevent immune response after transplantation, the grafted cells for treatment are preferably autologous cells established from the patient. However, if establishment, differentiation, preparation, etc., of iPS cells from patient-derived cells take a long time and such duration is considered undesirable to increase therapeutic effects, allogeneic or xenogeneic cells may be used in the present invention. In this case, it is preferable to perform blood type matching, HLA typing, etc., and use cells that are most unlikely to be rejected. From this point of view, it is desirable to prepare a bank of allogeneic iPS cells derived from many donors with different HLA types. It is more preferable to prepare any or all banks of the following: cells obtained by differentiation from such allogeneic iPS into cell types suitable for transplantation (e.g. chondrocytes); tissues for transplantation comprising such cells (e.g., three-dimensionally cultured tissues); such cells and tissues into which a therapeutic gene (e.g., IL-12) has been introduced; and graft materials comprising such cells and/or tissues. If such a bank is prepared, a graft material can be promptly provided to a patient in need of the gene therapy (e.g., a cancer patient).

After the graft material has been transplanted into a patient, if the expression of the transgene becomes unnecessary or a certain side effect is observed, the transplanted cells can be removed from the patient. From this point of time, the production of the secreted protein, which is a product of the transgene, can be eliminated. To ensure this, the transplanted cells are preferably in the form of a solid or tissue shape. Examples thereof include tissue comprising chondrocytes, and chondrocyte tissue three-dimensionally cultured using a scaffold.

When cells derived from iPS cells are used for transplantation, the transplanted cells may become cancerous, which has been a so-called major impediment in regenerative medicine. For example, even when the transplantation is performed after the iPS cell-derived cells are differentiated into cartilage, if only a small proportion of iPS-like undifferentiated cells are contained, teratomas may be generated from the cells after transplantation. To prevent this problem, the cells that form the graft material are preferably transplanted after the cells are irradiated to lose their growth potential. This irradiation may be performed immediately before the graft material is transplanted into a patient. More preferably, however, irradiation is performed after terminal differentiation into cells for transplantation and before preparation of a graft material. The irradiation conditions suitable for this purpose are provided in the present invention. More specifically, when soft X-rays are used, the irradiation dose is preferably 15 to 80 Gy, more preferably 20 to 40 Gy, and particularly preferably 30 to 40 Gy. For example, gamma rays may also be used instead of soft X-rays. In this case, the irradiation dose can be determined in terms of dose equivalents.

The cells to be transplanted are preferably differentiated into a cell type suitable for transplantation. The site suitable for implantation may vary depending on both the disease and the therapeutic gene. Accordingly, another feature of the present invention is that the implantation site and cell type can be suitably selected according to the purpose (because iPS cells can be induced to differentiate into various cells). For example, in cytokine gene therapy for melanoma, when cells having a therapeutic cytokine (e.g., IL-12) gene introduced thereinto are to be transplanted under the skin close to a tumor, cells that are considered to be easily engrafted subcutaneously, such as fibroblasts, can be selected.

It is generally preferable that the cells to be transplanted are differentiated into, for example, cartilage. This is because cartilage is avascular tissue in itself and does not require a high partial pressure of oxygen. Accordingly, even when the implantation site has a poor vascular blood flow with poor formation of new blood vessels, the transplanted cells can survive in the site for a long period of time. Furthermore, it is relatively easy for iPS cells to be induced into cartilage. Cartilage tissue is distinguishable from other tissues based on shape and hardness; furthermore, cartilage tissue can be three-dimensionally cultured on a scaffold. Accordingly, after induced cartilage tissue or three-dimensionally cultured cartilage tissue has been transplanted into a patient, if the transplanted cells need to be removed because the expression of the introduced gene becomes unnecessary or a certain side effect is observed, the graft can be relatively easily removed from the implantation site. Chondrocytes are expected to survive in vivo for a relatively long period of time without cell division. In addition, chondrocytes are relatively resistant to radiation, whereas cells with high growth potential, such as iPS cells, are susceptible to radiation. Accordingly, it is expected that radiation can ensure long-term survival without cell division and continuous expression of the introduced secreted protein gene.

The methods for introducing the gene include, for example, a method of infection with a viral vector, such as a retroviral vector, an adenoviral vector, a lentiviral vector, or an adeno-associated viral vector; and a method of transfection of a plasmid vector, an episomal vector, or the like using a non-viral vector, such as a cationic liposome, a cationic polymer, or electroporation. RNA can also be introduced. All the above gene transfer means are collectively referred to herein as vectors.

When a drug selection marker gene (conferring resistance to puromycin, blasticidin S, neomycin, hygromycin, etc.) is introduced with a therapeutic gene and then drug selection is performed, cells expressing the therapeutic gene can be selected and used.

In a preferable embodiment, a specific method for preparing cells for transplantation according to the present invention, particularly the timing of introducing the gene, can be suitably selected from various choices according to the purpose, case, etc. For example, in the case where there is relatively ample time before commencement of treatment, iPS cells can be newly derived from the patient's somatic cells (for example, fibroblasts) and differentiated into cells used for graft materials. In this case, vectors having a gene for a therapeutic purpose (for example, IL-12) and a drug selection marker gene (for example, puromycin-resistant gene) are introduced simultaneously with Oct-3/4, Sox2, Klf-4, etc., into patient-derived somatic cells. While iPS cells are induced from such cells and further differentiated into cells for transplantation (for example, chondrocytes), drug selection is continuously performed, whereby chondrocytes producing IL-12 are considered to be selectable. An advantage of this embodiment is that both a reprogramming gene and a therapeutic gene can be introduced in a single introduction, whereas a disadvantage thereof is that the expression of the therapeutic gene may be suppressed (silenced). Alternatively, cells for transplantation may be prepared by a method comprising first establishing iPS cells from patient-derived somatic cells and then introducing a therapeutic gene and a drug selection marker gene, followed by drug selection and differentiation induction. This method is preferable due to the low possibility of silencing, and is particularly advantageous for use when two or more cells that express different therapeutic genes in one patient are to be prepared. Alternatively, cells for transplantation can also be prepared by a method comprising first establishing iPS cells from patient-derived somatic cells, then inducing differentiation, and thereafter introducing a therapeutic gene and a drug selection marker gene, followed by drug selection and further induction of differentiation. This method is preferable due to the low possibility of silencing, and is advantageous for use when two or more cells that express different therapeutic genes in one patient are to be prepared. When it is necessary to hasten the onset of therapy and when patient-derived iPS cells cannot be used, allogeneic or xenogeneic iPS cell-derived cells can be used. Assuming that such cases may occur, a bank of allogenic iPS cells derived from many donors with different HLA types is preferably prepared. From such a bank, iPS cells that match the patient's HLA are selected and a therapeutic gene is introduced thereinto, followed by drug selection and differentiation induction, whereby cells for transplantation can be prepared. More preferably, for frequently occurring diseases such as cancers, if allogenic iPS cell-derived cells that are derived from many donors with different HLA types and that have a therapeutic gene, such as IL-12, introduced thereinto, are prepared as graft materials to establish a bank, such cells can be relatively quickly used for therapy after HLA typing, etc. Further, if allogeneic iPS cell-derived cells that are derived from many donors with different HLA types and that have a therapeutic gene, such as IL-12, introduced thereinto, are further induced to differentiate into cells suitable for transplantation, such as chondrocytes, such cells can be used as a graft material bank. Further, if allogeneic iPS cell-derived cells that are derived from many donors with different types of HLA and that have a therapeutic gene, such as IL-12, introduced thereinto are induced to differentiate into cells suitable for transplantation, such as chondrocytes, and then irradiated, such cells can be used as a graft material bank.

The iPS cells used in the present invention may be any cells reprogrammed or de-differentiated from the patient's somatic cells by some means, and do not need to have pluripotency in the strict sense of the word. Accordingly, the cells do not have to be iPS cells in the narrow sense of the word. For example, the iPS cells may be mesenchymal stem cell-like cells de-differentiated from somatic cells. De-differentiation as used herein means all the cellular changes in directions different from cell differentiation during normal ontogeny. Preferably, the cells to be used have the ability to differentiate into cells for transplantation (for example, chondrocytes).

### EXAMPLES

Examples are shown below; however, the present invention is not limited to these Examples.

### Example 1

Gene transfection into mouse iPS cell-derived chondrocytes during differentiation, and gene transfection into primary rabbit chondrocytes were performed. In accordance with the method of Takahashi and Yamanaka (Non-Patent Literature Cell. 2006, 25; 126(4): 663-76), C57B1/6 mouse fibroblasts were infected with a retroviral vector including Oct-3/4, Sox2, Klf-4, and c-Myc to establish iPS cells. The mouse iPS cells were cultured for 5 days using a low-adherent culture dish in a dMEM culture medium containing BMP2 (10 ng/mL) purchased from R&D Systems, Inc., TGF beta 1 (2 ng/mL) purchased from PeproTech Inc., and FBS (10%), thereby forming embryoid bodies. The thus-obtained embryoid bodies were cultured for 15 days on a gelatin-coated culture dish in the presence of BMP2, insulin (1 μg/mL) purchased from Sigma-Aldrich Co., and ascorbic acid (50 μg/mL) purchased from Nacalai Tesque, Inc. The cells were then infected with an amphotropic retroviral vector containing an EGFP expression unit, using a Retro Virus Packaging Kit Ampho purchased from Takara Bio Inc. by following the preparation procedure. Packaging cells (GT3hi) were transfected with three types of vectors, i.e., pGP vector, pE-ampho, and pDON-5 GFP Neo, using the calcium phosphate method. The culture supernatant 24 to 48 hours after the transfection was collected as a retroviral stock solution. A 24-well culture plate was coated with RetroNectin purchased from Takara Bio Inc. at a concentration of 50 μg/mL to prepare a RetroNectin-coated plate. A 2-fold diluted retroviral stock solution was added to the prepared plate, and virions were adsorbed thereto. Subsequently, cartilage precursor cells differentiated from 1×10⁵ mouse iPS cells or rabbit chondrocytes obtained from the knee joint of white rabbits were seeded. The cells were thereafter cultured for 3 days under chondrocyte-inducing conditions to be differentiated into chondrocytes. Observation was performed under a differential interference contrast microscope.

### Example 2

FIG. 2 shows the results of the experiment in Example 1. In regard to the mouse iPS cell-derived chondrocytes and primary rabbit chondrocytes, FIG. 2 shows differential interference contrast (DIC) microscope images of two different fields and fluorescence microscope images (NIBA) of these two fields. The arrows in the fluorescence microscope images indicate EGFP expression cells. The results show that gene transfection during differentiation of the mouse iPS cells into chondrocytes results in gene expression with higher efficiency, compared to the case of gene transfection into chondrocytes from rabbit cartilage.

### Example 3

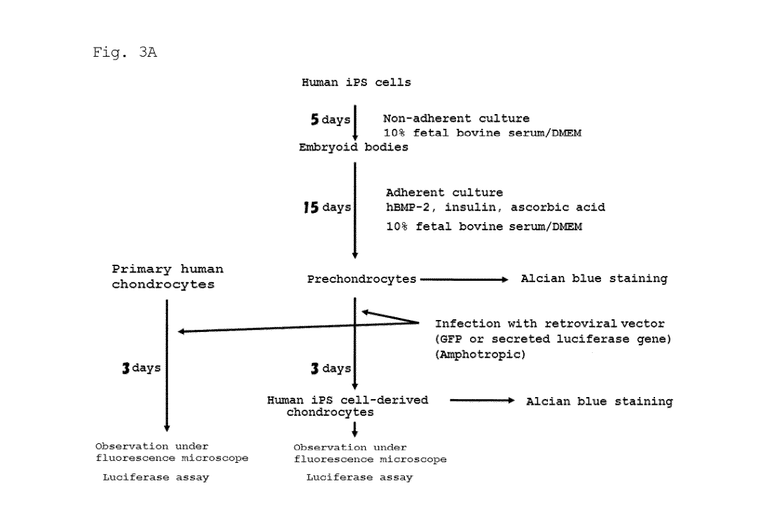

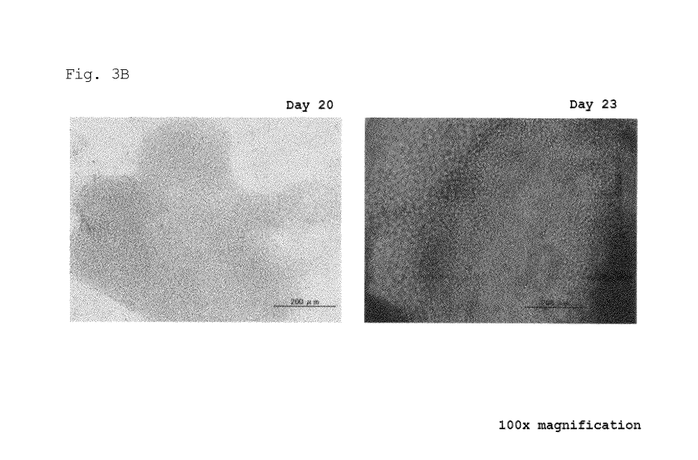

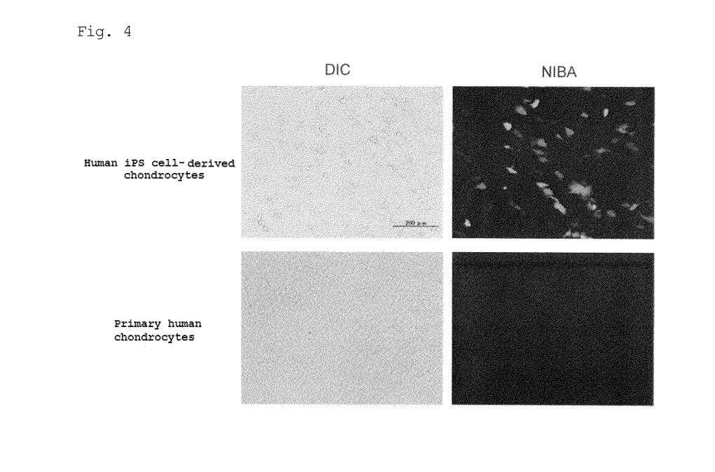

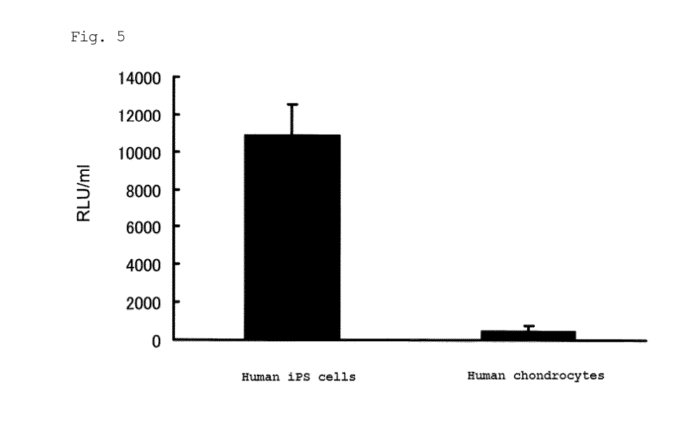

Gene transfection and expression efficiency between the case when human iPS cell-derived chondrocytes are infected with a retrovirus during differentiation and the case when primary human chondrocytes are infected with the retrovirus were compared. FIG. 3A shows a summary of the experiment. Keratinocytes were transfected with a plasmid vector containing Oct-3/4, Sox2, Klf-4, c-Myc, and Lin28 to establish iPS cells. These human iPS cells were cultured for 5 days using a low-adherent culture dish in a DMEM culture medium containing FBS (10%), thereby forming embryoid bodies. The thus-obtained embryoid bodies were cultured for 18 days on a gelatin-coated culture dish in the presence of BMP2, insulin (1 μg/mL), and ascorbic acid (50 μg/mL). Some of the cells was washed twice with PBS(−) and then once with a 3% acetic acid solution on day 20 and day 23 of culturing. Subsequently, an alcian blue staining solution (pH of 2.5) manufactured by Nacalai Tesque, Inc. was added to stain the cells for 1 hour at room temperature. After washing with PBS(−) three times, the cells were observed under a microscope. An image stained with blue was observed in the cells of day 20 (FIG. 3B). The staining was stronger on the cells of day 23 than the cells on day 20, and it was confirmed that the induction of differentiation into cartilage occurred intensively from day 20 and day 23 in this system.

The cells on day 15 of culturing were infected with an amphotropic retroviral vector containing an EGFP expression unit or secreted luciferase expression unit, using a Retro Virus Packaging Kit Ampho purchased from Takara Bio Inc. by following the preparation procedure. Three types of vectors, i.e., pGP vector, pE-ampho, and pDON-5 GFP Neo or pDON-5 Luc2 Neo, were transfected into packaging cells (GT3hi) using the calcium phosphate method. The culture supernatant 24 to 48 hours after the transfection was collected as a retroviral stock solution. A 24-well culture plate was coated with RetroNectin purchased from Takara Bio Inc. at a concentration of 50 μg/mL to prepare a RetroNectin-coated plate. A 2-fold diluted retroviral stock solution prepared using pDON-5 GFP Neo was added to the prepared plate, and allowed to stand for 4 hours at room temperature to adsorb virions. Subsequently, cartilage precursor cells differentiated from 1×10⁵ human iPS cells or primary human chondrocytes were seeded. The cells were thereafter cultured for 3 days under chondrocyte-inducing conditions to be differentiated into chondrocytes. Observation was made under a differential interference contrast microscope.

### Example 4

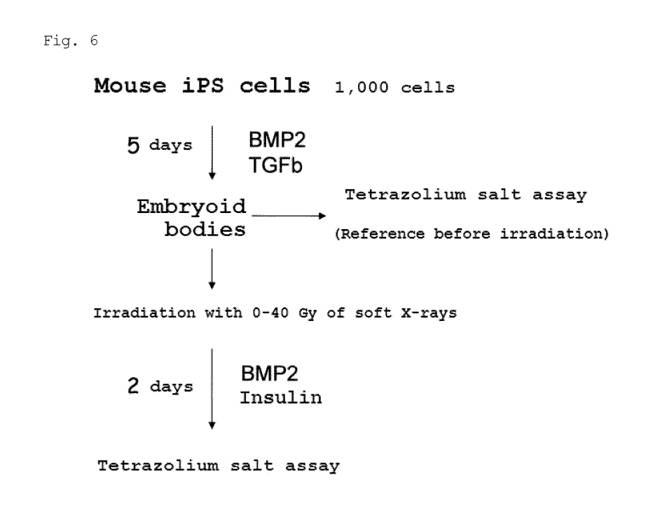

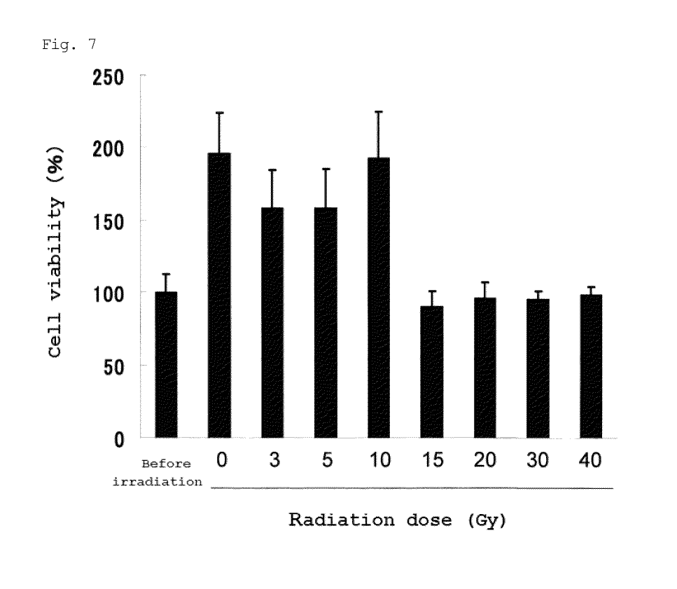

FIG. 4 shows the results of the experiment shown in FIG. 3A. FIG. 4 shows differential interference contrast microscope images (left) and fluorescence microscope images (right) of the cells obtained by introducing a GFP gene into human iPS cells during differentiation and further differentiating the cells into chondrocytes (above), and the primary human chondrocytes transfected with a GFP gene (below). In the former, GFP is efficiently introduced and expressed; however, almost no expression is observed in the latter. Therefore, it is clear from the GFP expression that infection of human iPS-derived chondrocytes with a retrovirus during differentiation results in very high gene transfection and expression efficiency, compared to the case of the infection of primary human chondrocytes with a retrovirus.

### Example 5

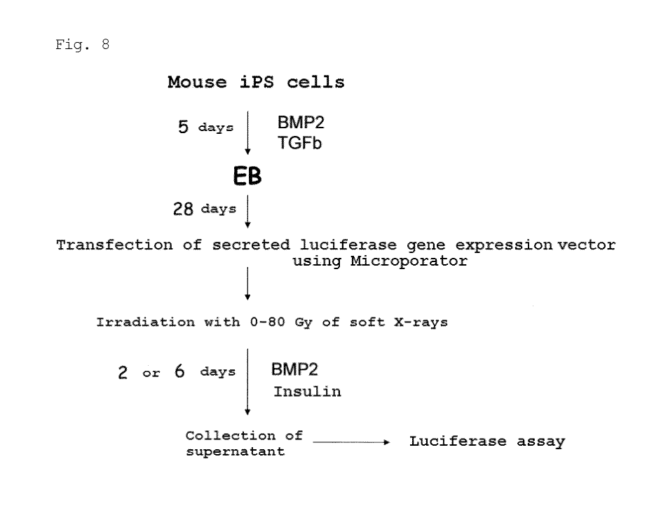

In the same manner as in FIG. 3A, virions were adsorbed using a retroviral stock solution prepared using pDON-5 GFP Neo. Subsequently, cartilage precursor cells differentiated from 1×10^{r} human iPS cells or primary human chondrocytes were seeded. The cells were thereafter cultured for 3 days under chondrocyte-inducing conditions, and the culture supernatant was collected. The luciferase activity in the culture supernatant was measured using a Ready-To-Glow Dual Secreted Reporter Assay Kit produced by Clontech Laboratories, Inc. FIG. 5 shows the results. It is clear from the luciferase expression that also in humans, gene transfection during the differentiation of iPS cells into chondrocytes via embryoid bodies results in gene expression with high efficiency.

### Example 6

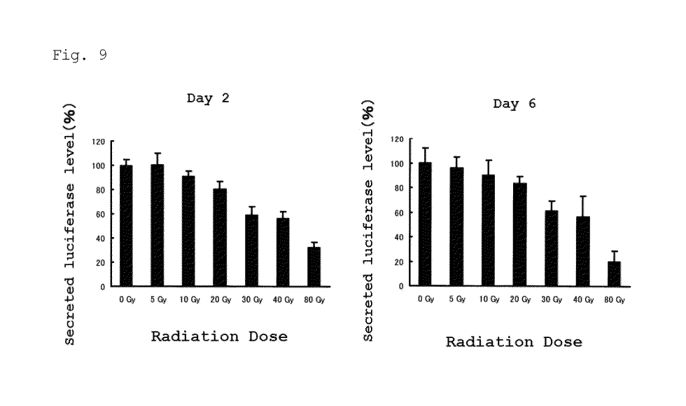

FIG. 6 is a summary of an experiment in which chondrocytes that were differentiated from iPS cells are irradiated with soft X-rays to examine the influence of the dose on the cell growth. Mouse iPS cells were cultured for 5 days in the presence of BMP2 and TGFβ to form embryoid bodies. The thus-obtained embryoid bodies were irradiated with various doses (0 to 40 Gy) of soft X-rays, and then cultured for 2 days on a gelatin-coated culture dish in the presence of BMP2 and insulin. A cell count reagent produced by Nacalai Tesque, Inc. was added to these cells for 2 hours, and subsequently, OD450 was measured (tetrazolium salt assay). As a reference before irradiation, a cell count reagent produced by Nacalai Tesque, Inc. was added for 2 hours to embryoid bodies not irradiated with soft X-rays, as shown in FIG. 3, and OD450 was thereafter measured.

### Example 7

FIG. 7 shows the results of Example 6. The values on the vertical axis (the cell viability (%)) were determined from the following formula.

Cell viability(%)=(OD450 of the cells in each group)/(OD450 of the reference before irradiation)*100

The cells irradiated with soft X-rays with a radiation dose of 3 to 10 Gy showed cell growth comparable to that of non-irradiated cells. However, it was found that the growth was almost completely inhibited in the cells irradiated with a dose of 15 Gy or more.

### Example 8

FIG. 8 is a summary of an experiment to observe the influence of the dose of soft X-rays on a plasmid vector introduced into chondrocytes differentiated from iPS cells. Mouse iPS cells were cultured for 5 days in the presence of BMP2 and TGFβ to form embryoid bodies. The thus-obtained embryoid bodies were cultured for an additional 28 days, and subsequently, a pMetLuc2-Control vector (secreted luciferase gene expression vector) was introduced thereinto using a microporator. These cells were irradiated with various doses (0 to 80 Gy) of soft X-rays, and the cells were then cultured for an additional 2 days or 6 days in the presence of BMP2 and insulin. The culture supernatants of these cells were collected and used in a luciferase assay.

### Example 9

FIG. 9 shows the results of Example 8. The values on the vertical axis (the amount of secreted luciferase production (%)) were determined from the following formula.

Amount of luciferase production(%)=(RLU in the cell culture supernatant of each group)/(RLU in the cell culture supernatant of the non-irradiated group(0 Gy))*100

It was found that the amount of secreted luciferase production decreased in a manner dependent on the amount of secreted luciferase production. It was also found that the amount of secreted luciferase production significantly decreased when the cells were irradiated with a high dose that exceeds 40 Gy.

### Example 10

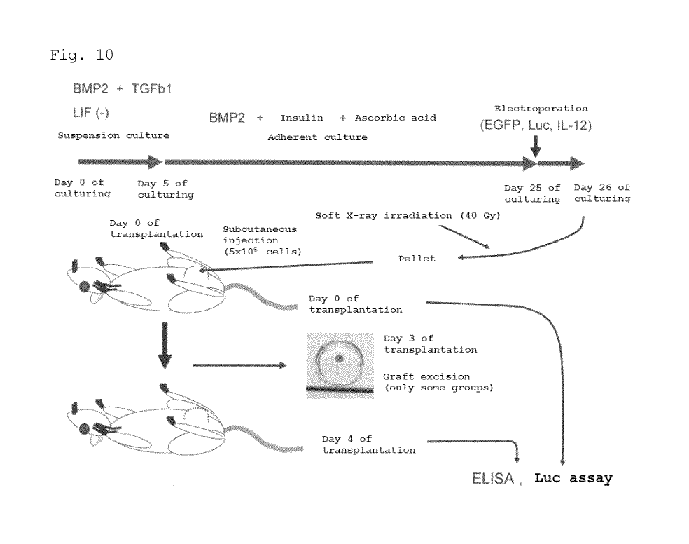

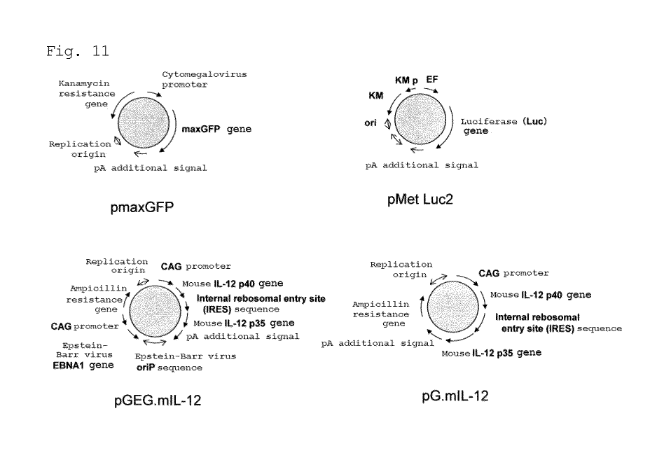

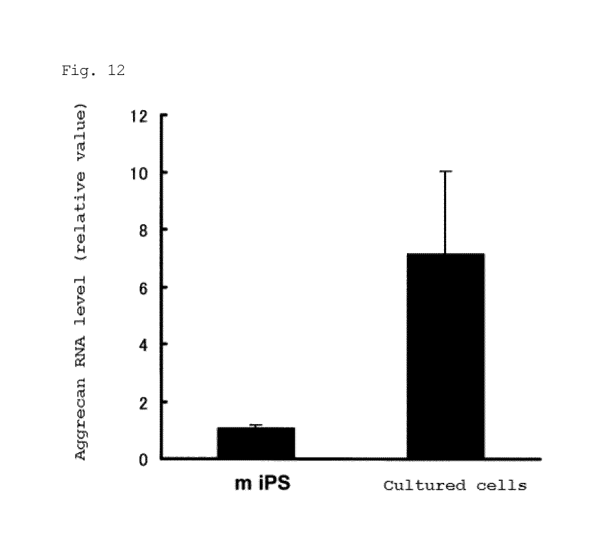

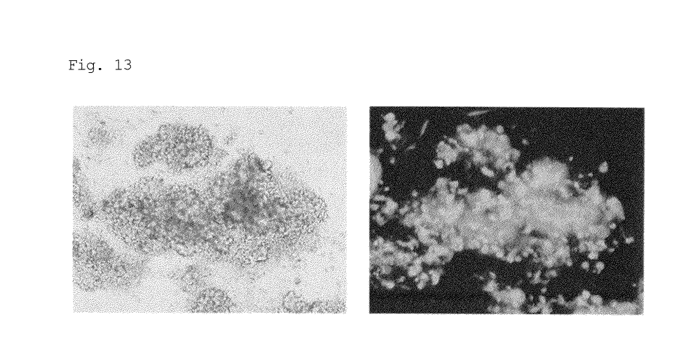

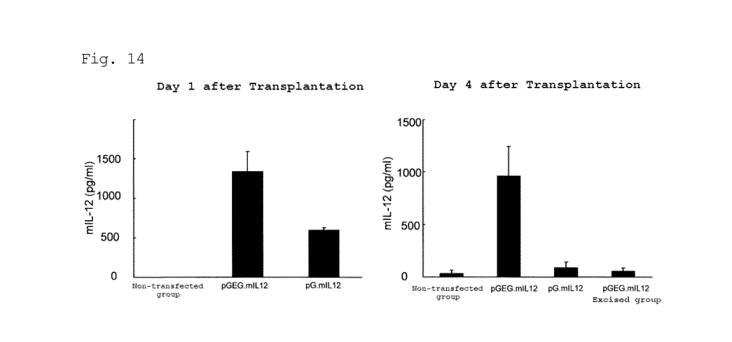

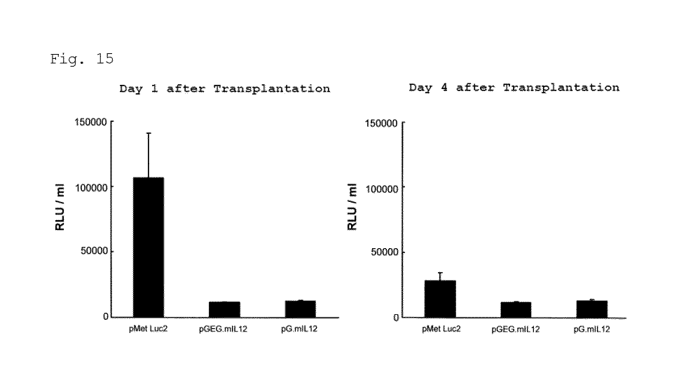

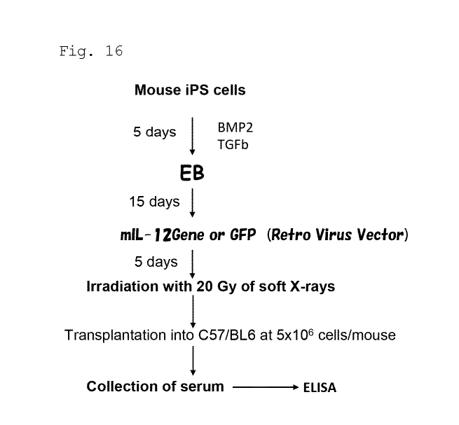

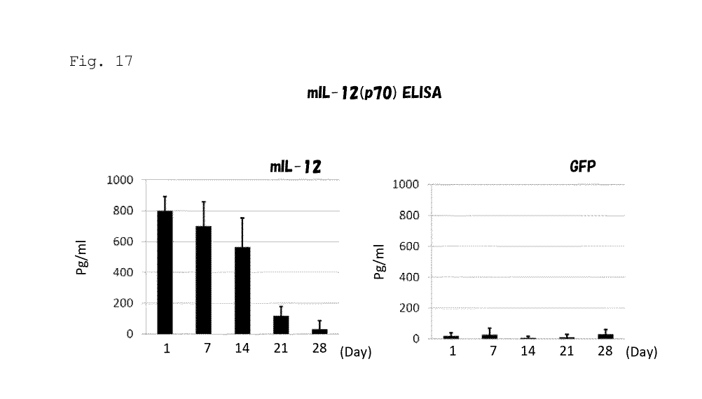

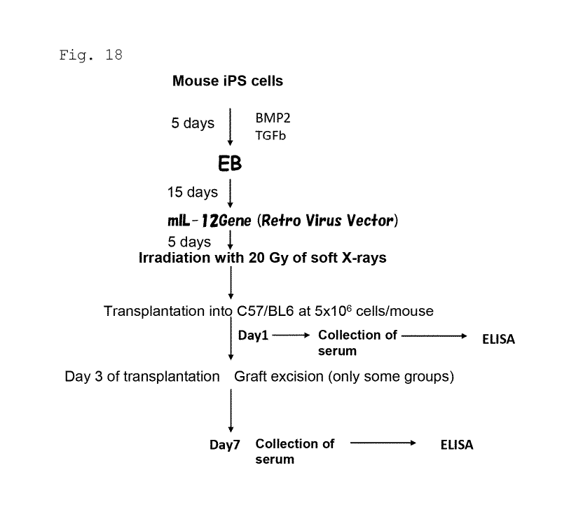

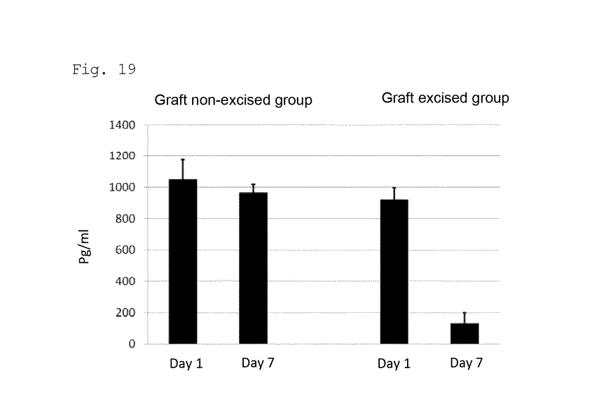

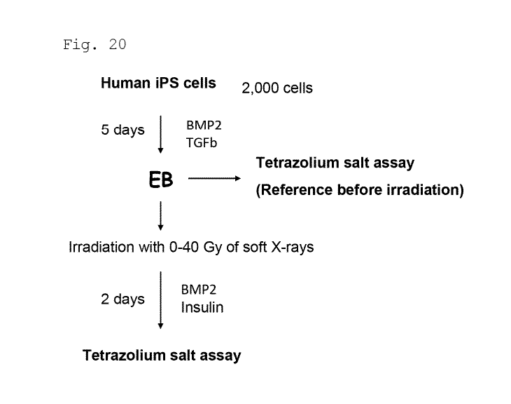

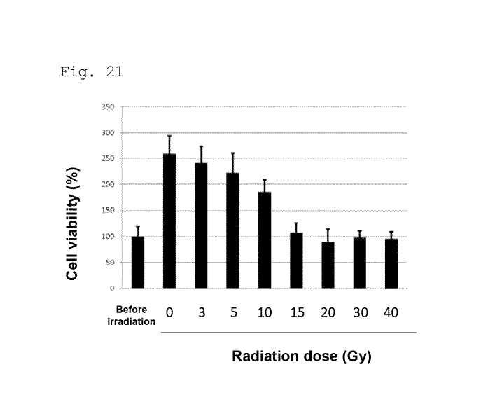

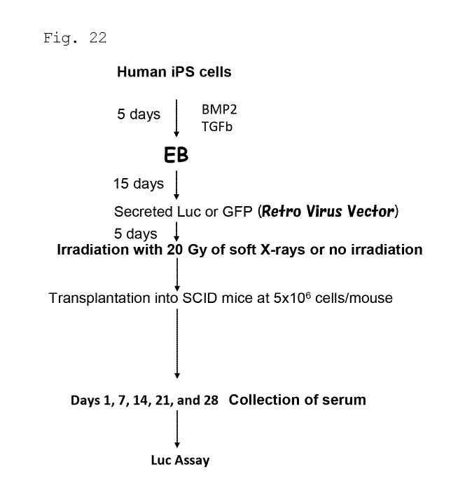

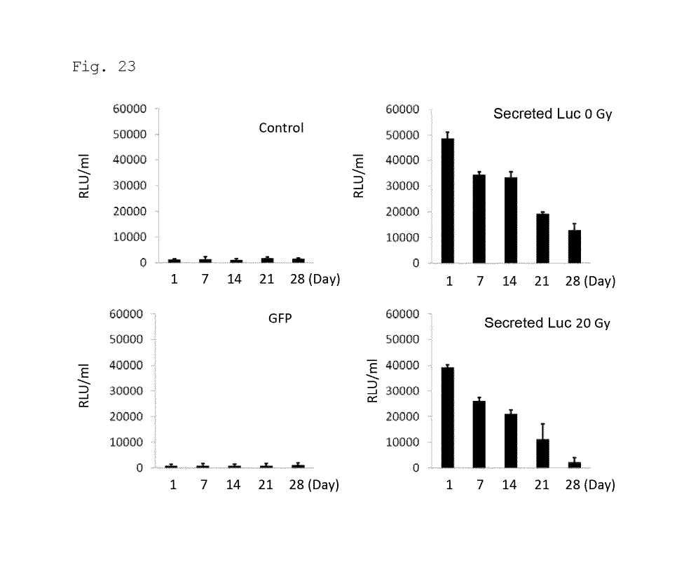

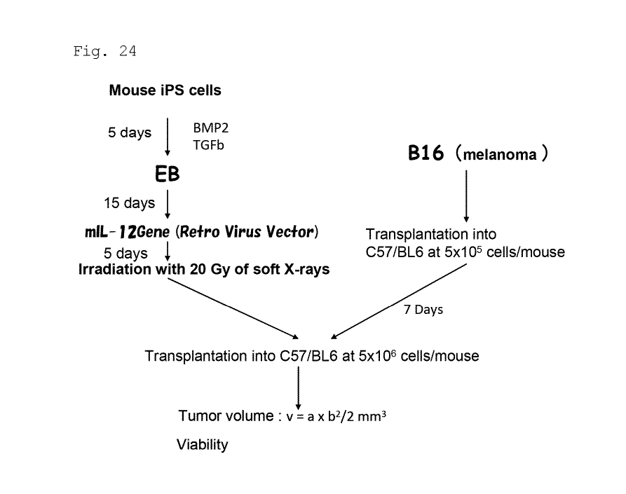

FIG. 10 shows a summary of a transplant experiment. Mouse iPS cells were cultured for 5 days on a low-adherent culture dish in a culture medium containing BMP2 and TGFbeta1 but free of LIF. Subsequently, the cells were cultured for 20 days on an adherent culture dish in a culture medium containing BMP2, insulin, and ascorbic acid. These cells were divided into three groups, and the groups were transfected with three different plasmid vectors respectively containing EGFP, secreted Luc, and IL-21, using electroporation. As the control, cells that were simply electroporated without gene transfection were prepared. The following day, each cell was lifted with trypsin and irradiated with 40 Gy of soft X-rays. Subsequently, the cells were centrifuged, the supernatant was discarded, and the pellet was collected by a syringe. Then, C57BL/6 mice were subcutaneously injected with 5,000,000 cells per mouse. On the following day and 4 days later, blood was collected from a tail vein of the mice, and the serum was adjusted and used for ELISA and a Luc assay. Further, some of the mice transplanted with the cells transfected with the IL-12 gene underwent the dissection of the transplantation site on day 3 after transplantation, and the tissue containing the injected cells was excised. Subsequently, blood was collected on day 4 after transplantation in the same manner described above.

### Example 11

FIG. 11 shows the plasmid vectors used in the experiment in Example 10. pMaxGFP was purchased from Amaxa Biosystems, and pMetLuc2 was purchased from Clontech Laboratories, Inc. pGEG.mIL-12 and pG.mIL-12 are described in Non-Patent Literature (Asada, H. et al., Mol. Ther. 5 (5): 609-616, 2002).

### Example 12

The total RNA was collected from iPS cells and the cells on day 25 of culturing in Example 10, and real time RT-PCR was performed using a primer and probe specific to aggrecan. FIG. 12 shows the results. The expression of aggrecan was increased in the cells on day 25 of culturing in Example 1, compared to iPS cells. This shows that the cells were differentiated into cartilage-like cells.

### Example 13

The left image in FIG. 13 shows a differential interference contrast microscope image of the cells on day 25 of culturing described in Example 10. Chondrocyte-like cell masses were observed. The right image in FIG. 13 shows a fluorescence microscope image of the same cells one day after transfection with pmaxGFP as described in Example 1. Green fluorescence of GFP was observed in 90% or more of the cells. This reveals intense expression of the transfected gene.

### Example 14

In vivo IL-12 gene expression was examined. The levels of IL-12 p70 in the serum collected from the mice on day 1 and day 4 after transplantation described in Example 10, was measured using an IL-12 p70 ELISA kit purchased from R&D Systems Science. FIG. 14 (both left and right) shows the results. It became clear from the results that the group transfected with pGEG.mIL-12 or pG.mIL-12 showed a significant increase in the levels of IL-12 p70 in the serum, compared to the group that was not subjected to gene transfection; the group transfected with pGEG.mIL-12 had higher levels of serum IL-12 p70 than the group transfected with pG.mIL-12; and the group from which the transplanted tissue was excised on day 3 after transplantation of pGEG.mIL-12-transfected cells showed a decrease in the IL-12 p70 serum levels.

### Example 15

In vivo luc expression was examined. FIG. 14 shows the luc activity in the serum collected from the mice on day 1 (left) and day 4 (right) after transplantation described in Example 10. The results show that the group transfected with pMetLuc2 had a significant increase in the luc activity in the serum, compared to the group transfected with the IL-12 gene.

### Example 16

Mouse iPS cells were suspension-cultured in the presence of mouse recombinant TGFβ and human recombinant BMP2 using a lipidure-coat plate manufactured by NOF Corporation, thereby forming embryoid bodies. Subsequently, the embryoid bodies were maintained in adherent culture in the presence of human recombinant BMP2, ascorbic acid, and insulin for 15 days, thereby preparing cartilage precursor cells. The cartilage precursor cells were infected with a mouse IL-12- or GFP-expressing retroviral vector prepared using a Platinum Retroviral Expression System and cultured thereafter for 5 days. On day 5 of culturing, the cells were irradiated with 20 Gy of soft X-rays, and iPS cell-derived chondrocytes (5×10⁶) were transplanted. The serum was collected on day 1, day 7, day 14, day 21, and day 28, and the IL-12 levels in the serum were measured using a mouse IL-12 ELISA kit manufactured by R&D Systems. FIG. 17 shows the results.

### Example 17

1,000 mouse iPS cells as a mass per well were suspension-cultured in the presence of mouse recombinant TGFβ and human recombinant BMP2 using a lipidure-coat plate (A-U96) manufactured by NOF Corporation, thereby forming embryoid bodies. Subsequently, the embryoid bodies were maintained in adherent culture in the presence of human recombinant BMP2, ascorbic acid, and insulin for 15 days, thereby preparing cartilage precursor cells. The cartilage precursor cells were infected with a mouse IL-12 or GFP-expressing retroviral vector prepared using a Platinum Retroviral Expression System and cultured thereafter for days. On day 5 of culturing, the cells were irradiated with 20 Gy of soft X-rays, and iPS cell-derived chondrocytes (5×10⁶) were transplanted. On day 3 after transplantation, the cells were divided into a group that underwent excision of transplanted cartilage masses and a group that did not undergo such excision. One day after transplantation and day 7 after transplantation, the serum was collected from both groups and the serum IL-12 levels were measured using a mouse IL-12 ELISA kit manufactured by R&D Systems. FIG. 19 shows the results.

### Example 18

1,000 mouse iPS cells as a mass per well were suspension-cultured in the presence of mouse recombinant TGFβ and human recombinant BMP2 using a lipidure-coat plate (A-U96) manufactured by NOF Corporation, thereby forming embryoid bodies. Subsequently, these cells were irradiated with 0 G, 3 Gy, 5 Gy, Gy, 15 Gy, 20 Gy, 30 Gy, and 40 Gy of soft X-rays, and then maintained in adherent culture in the presence of human recombinant BMP2, ascorbic acid, and insulin for 2 days in a 96-well plate, and the cell viability was examined by monitoring the cell growth using a cell count reagent manufactured by Nacalai Tesque, Inc. FIG. 21 shows the results.

### Example 19

Human iPS cells (2,000/well) were suspension-cultured in the presence of mouse recombinant TGFβ and human recombinant BMP2 using a lipidure-coat plate (A-U96) manufactured by NOF Corporation, thereby forming embryoid bodies. Subsequently, the embryoid bodies were maintained in adherent culture in the presence of human recombinant BMP2, ascorbic acid, and insulin for 15 days, thereby preparing cartilage precursor cells. The cartilage precursor cells were infected with a secreted luciferase (MetLuc2) or GFP-expressing retroviral vector prepared using a Platinum Retroviral Expression System and cultured thereafter for 5 days. On day 5 of culturing, the cells were divided into a group irradiated with 20 Gy of soft X-rays and a non-irradiated group. Human iPS cell-derived chondrocytes (5×10⁶) were subcutaneously transplanted into immune-deficient mice (SCID mice). The serum was collected on day 1, day 7, day 14, day 21, and day 28, and the secreted luciferase was measured. FIG. 23 shows the results.

### Example 20

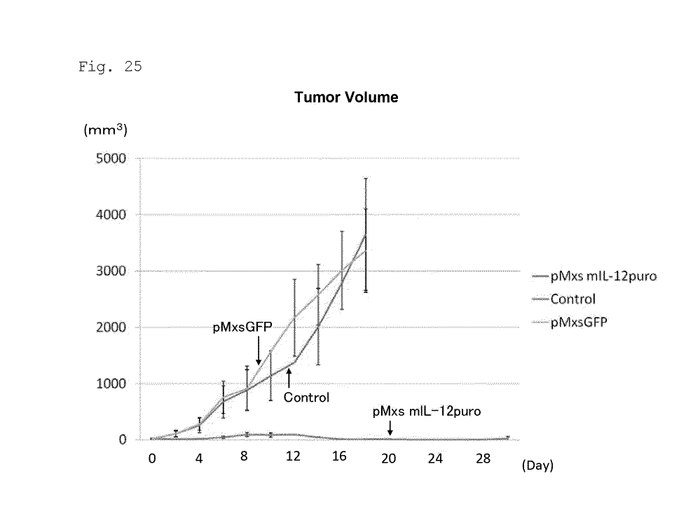

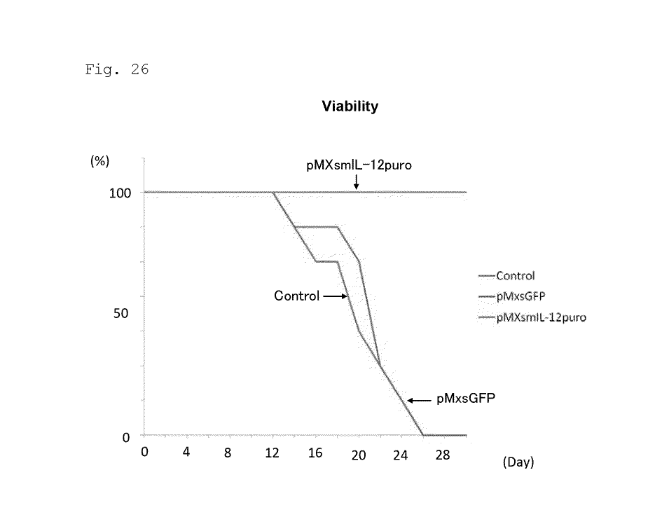

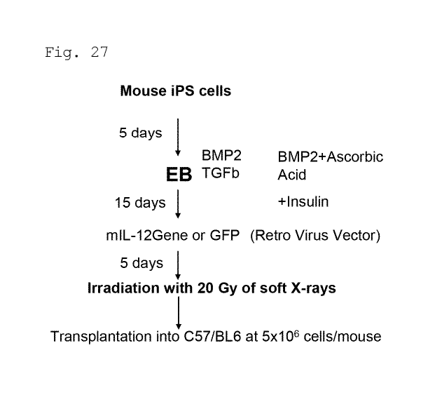

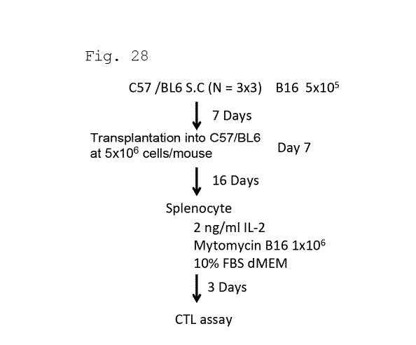

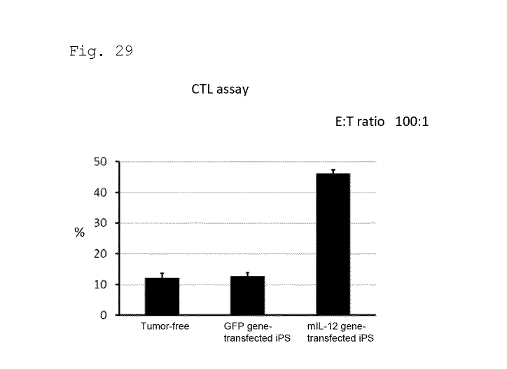

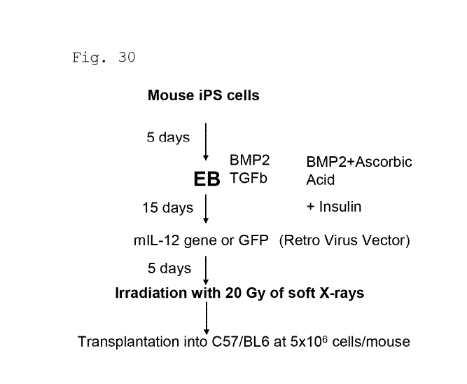

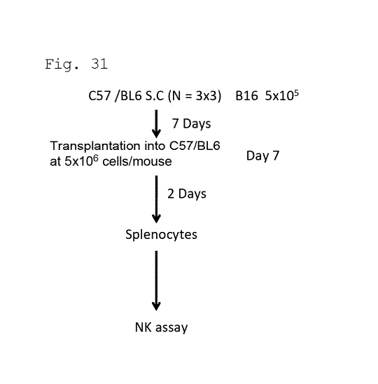

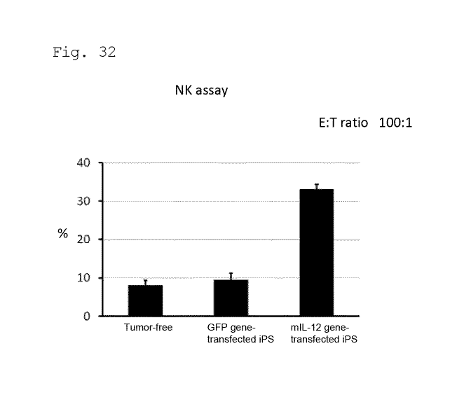

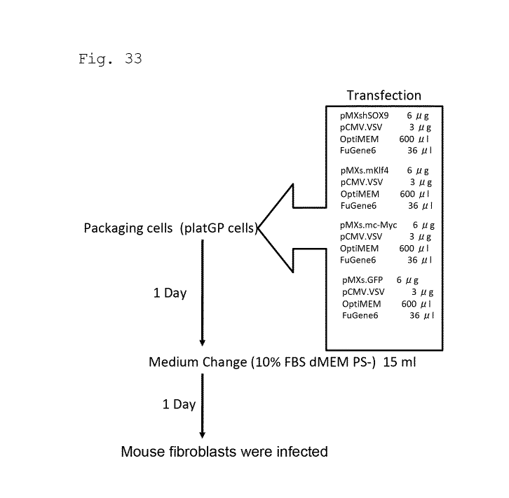

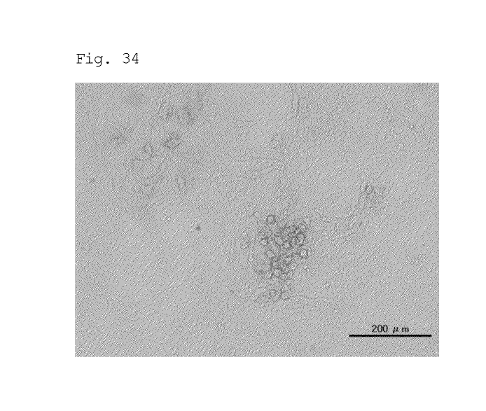

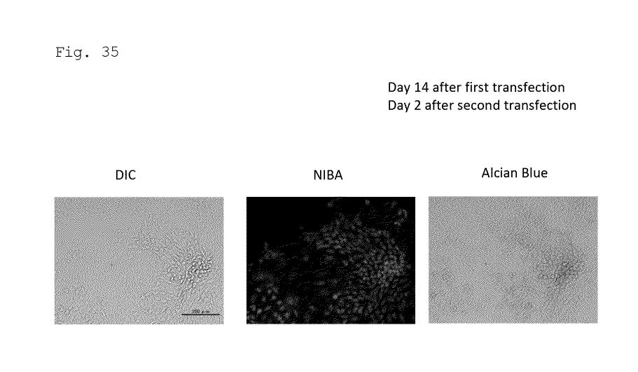

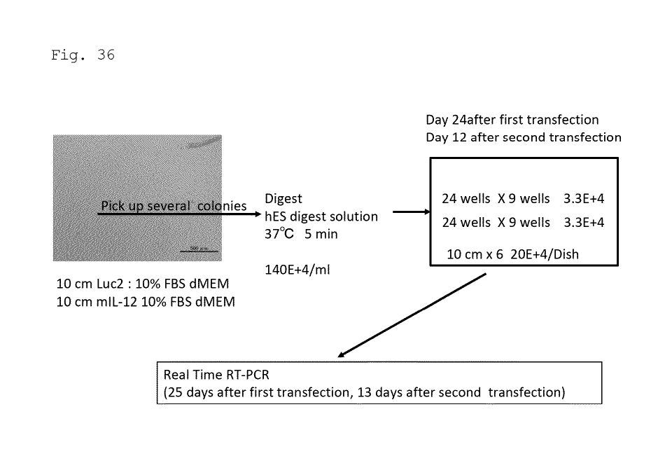

A mouse melanoma B16 cell line (5×10⁵ cells) was subcutaneously transplanted into C57BL/6 mice. Seven days later, tumor formation was confirmed, followed by transplantation of mouse iPS cell-derived chondrocytes (5×10⁶) infected with a mouse IL-12 gene expressing retroviral vector that was prepared using a Platinum Retroviral Expression System. The major axis and the minor axis of the tumor were measured every 2 days after tumor transplantation, and the volume was calculated from the measured values. For the calculation, the following formula was used: volume=(major axis×minor axis²)/2. FIG. 25 shows the results.

### Example 21

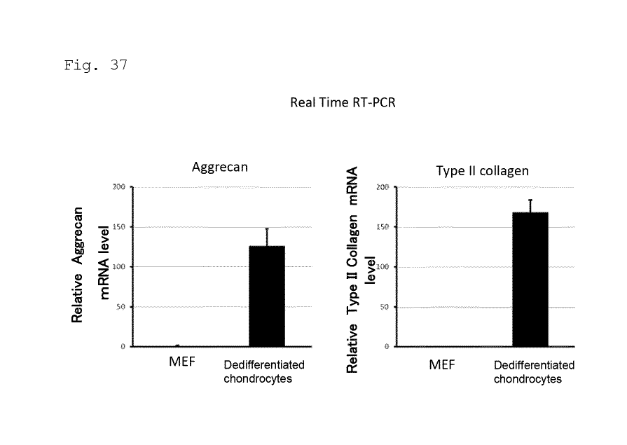

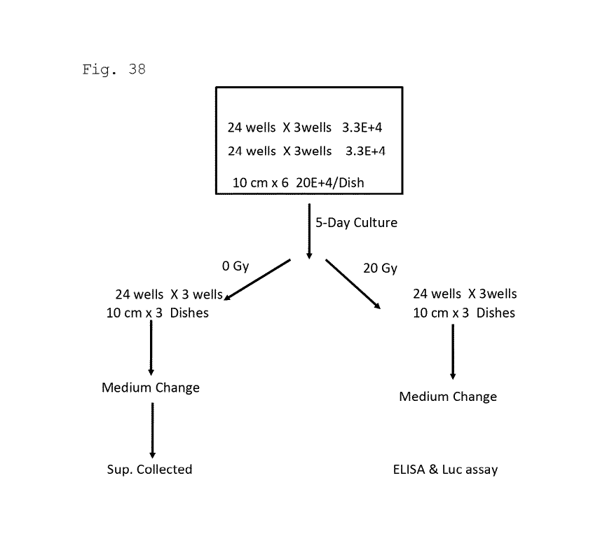

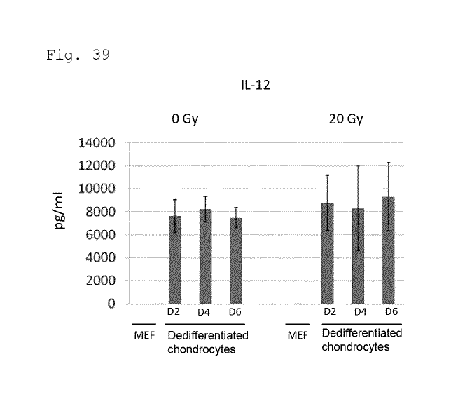

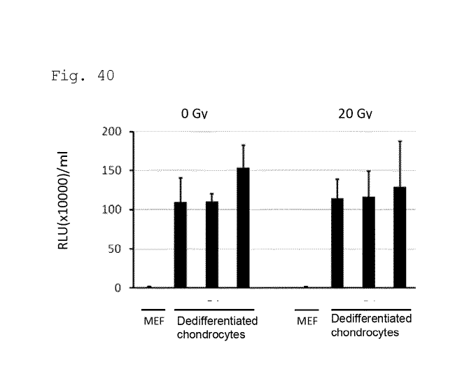

A mouse melanoma B16 cell line (5×10⁵ cells) was subcutaneously transplanted into C57BL/6 mice. Seven days later, tumor formation was confirmed, followed by transplantation of mouse iPS cell-derived chondrocytes (5×10⁵) infected with a mouse IL-12 gene expressing retroviral vector that was prepared using a Platinum Retroviral Expression System. The viability after tumor transplantation was examined. FIG. 26 shows the results.

### Example 22

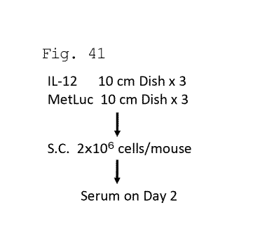

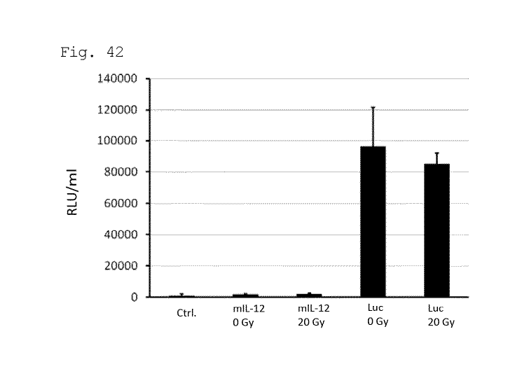

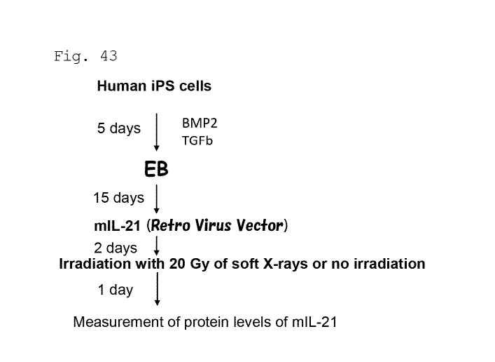

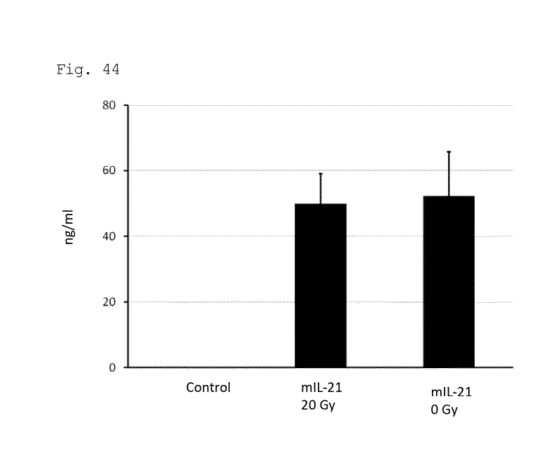

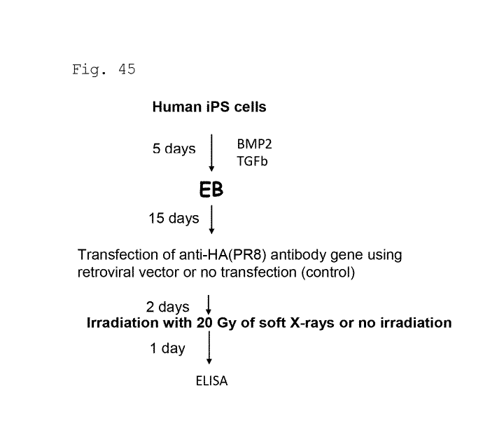

A mouse melanoma B16 cell line (5×10⁵ cells) was subcutaneously transplanted into C57BL/6 mice. Seven days later, tumor formation was confirmed, followed by transplantation of mouse iPS cell-derived chondrocytes (5×10⁶) infected with a retroviral vector containing mouse IL-12 gene, which was prepared using a Platinum Retroviral Expression System. Two days later, splenocytes were collected and used as effector cells, which were then mixed with Yaci cells labeled with Cr⁵¹ as the target cells at a 100:1 ratio. The mixture was cultured under conditions of 37° C. and 5% CO² for 4 hours, and the culture supernatant was collected. The γ dose was measured using a γ counter, and the CTL cell activity, i.e., a tumor-specific cell killing effect, was calculated from the measured value. FIG. 29 shows the results.

### Example 23

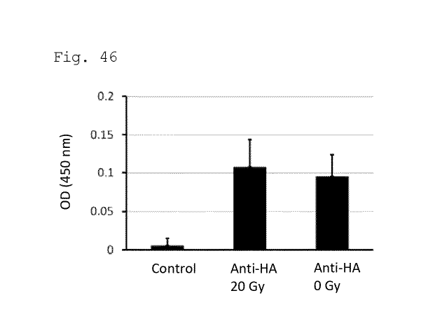

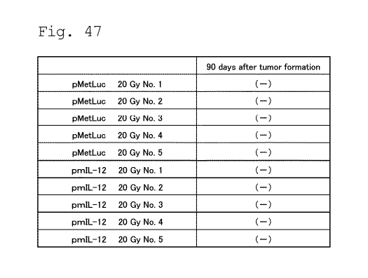

A mouse melanoma B16 cell line (5×10⁵ cells) was subcutaneously transplanted into C57BL/6 mice. Seven days later, tumor formation was confirmed, followed by transplantation of mouse iPS cell-derived chondrocytes (5×10⁵) infected with a mouse IL-12 gene expressing retroviral vector, which was prepared using a Platinum Retroviral Expression System. 16 days later, splenocytes were collected and co-cultured in the presence of mitomycin-treated B16 cells and 2 ng/mL of mouse recombinant IL-2 for 3 days to be used as effector cells. The effector cells were mixed with B16 cells labeled with Cr⁵¹ as the target cells at a 100:1 ratio, and the mixture was cultured under conditions of 37° C. and 5% CO² for 4 hours. Then, the culture supernatant was collected. The γ dose was measured using a γ counter, and the NK cell activity, i.e., a tumor non-specific cell killing effect, was calculated from the measured value. FIG. 32 shows the results.

### Example 24

Plat-GP packaging cells produced by Cell Biolabs, Inc. were co-transfected with a plasmid vector constructed by inserting a human Sox9 gene, mouse Klf4 gene, mouse cMyc gene, and Aequorea victoria-derived GFP gene into a pMXs puro vector produced by Cell Biolabs, Inc. and pCMV.VSV also produced by Cell Biolabs, Inc., using Fugene 6 manufactured by Roche Ltd. Two days after transfection, the culture supernatant was collected, supplemented with polybrene (final concentration: 4 μg/mL), and used to infect fetal mouse fibroblasts. On day 9 after infection, the cells were stained with alcian blue. FIG. 34 shows the results.

### Example 25

Plat-GP cells were co-transfected with a plasmid vector constructed by inserting a mouse IL-12 gene and a firefly-derived secreted luciferase (MetLuc2) gene into a pMXs puro vector, and pCMV.VSV, using Fugene 6, thereby producing a retroviral vector containing a mouse IL-12, MetLuc2, and GFP gene. On day 12 after the first gene transfection, the produced retroviral vector was used to infect dedifferentiated chondrocytes during differentiation induction, which were re-seeded onto a 10-cm culture dish at a cell count of 5×10⁵/dish on the day before infection. Two days after the second infection, cells transfected with a GFP gene were subjected to fluorescent observation and stained with alcian blue. FIG. 35 shows the results.

### Example 26

The total RNA was collected from the cells on day 13 after the second infection, using a QuickGene RNA cultured cell kit manufactured by Fujifilm Corporation. Subsequently, cDNA was synthesized using a High Capacity RNA to cDNA kit manufactured by Applied Biosystems, Inc. Real time RT-PCR was then performed using aggrecan, i.e., a chondrocyte-specific marker gene, and a TaqMan probe and primer set that targets the type II collagen gene. FIG. 37 shows the results.

### Example 27

A retroviral vector containing mouse IL-12 was produced. The produced retroviral vector was used to infect dedifferentiated chondrocytes during differentiation induction, which were re-seeded onto a 10-cm culture dish at a cell count of 5×10⁵/dish on the day before infection. The infection was performed on day 12 after the cells were infected with a retroviral vector containing an hSOX9, mKlf4, and mMyc gene. The cells were then cultured in dMEM containing 10% fetal bovine serum for 5 days after infection, and seeded onto a 24-well plate at a cell count of 3.3×10⁴ per well. The culture medium was replaced on day 1, day 3, and day 5. The cells were divided into a group irradiated with 20 Gy of soft X-rays and a non-irradiated group. The culture supernatant was collected on day 2, day 4, and day 6 after irradiation, and the mouse IL-12 levels were measured by ELISA. FIG. 39 shows the results.

### Example 28

A retroviral vector containing a secreted luciferase gene was produced. The produced retroviral vector was used to infect dedifferentiated chondrocytes during differentiation induction, which were re-seeded onto a 10-cm culture dish at a cell count of 5×10⁵/dish on the day before infection. The infection was performed on day 12 after the cells were infected with a retroviral vector containing an hSOX9, mKlf4, and mMyc gene. The cells were then cultured in dMEM containing 10% fetal bovine serum for 5 days after infection, and seeded onto a 24-well plate at a cell count of 3.3×10⁴ per well. The culture medium was replaced on day 1, day 3, and day 5. The cells were divided into a group irradiated with 20 Gy of soft X-ray and a non-irradiated group. The culture supernatant was collected on day 2, day 4, and day 6 after irradiation, and a luciferase assay was performed. FIG. 40 shows the results.

### Example 29

A retroviral vector containing a secreted luciferase gene was produced. The produced retroviral vector was used to infect dedifferentiated chondrocytes during differentiation induction, which were re-seeded onto a 10-cm culture dish at a cell count of 5×10⁵/dish on the day before infection. The infection was performed on day 12 after the cells were infected with a retroviral vector containing an hSOX9, mKlf4, and mMyc gene. The cells were then cultured in dMEM containing 10% fetal bovine serum for 5 days after infection, and 2×10⁶ cells were subcutaneously transplanted into C57BL/6 mice. Two days later, the serum was collected and a luciferase assay was performed. FIG. 42 shows the results.

### Example 30

Human iPS cells (2,000/well) were suspension-cultured in the presence of mouse recombinant TGFβ and human recombinant BMP2 using a lipidure-coat plate (A-U96) manufactured by NOF Corporation, thereby forming embryoid bodies. Subsequently, the embryoid bodies were maintained in adherent culture in the presence of human recombinant BMP2, ascorbic acid, and insulin for 15 days, thereby preparing cartilage precursor cells. The cartilage precursor cells were infected with a mouse IL-12 gene expressing retroviral vector prepared using a Platinum Retroviral Expression System. Subsequently, on day 2 of culturing, the cells were divided into a group irradiated with 20 Gy of soft X-rays and a non-irradiated group. The cells were cultured for 24 hours after irradiation, and the supernatant was collected. After staining using a mIL-21 FlowCytomix Simplex Kit manufactured by e-Bioscience, Inc., the protein levels of mIL-21 in the supernatant were measured using a FacsCalibur flow cytometer manufactured by Becton, Dickinson and Company. FIG. 44 shows the results.

### Example 31

Mouse splenocytes were suspended in an RPMI 1640 culture medium containing 10% fetal bovine serum. Subsequently, recombinant influenza H1N1 HA (A/Puerto Rico/8/1934), produced by Sino Biological Inc., was added thereto, and the cells were cultured for 5 days. The total RNA was extracted from the splenocytes, and a reverse transcription reaction was performed to synthesize cDNA. The cDNA sequence of the heavy chain of immunoglobulin was amplified by PCR using VH primer (5′-gaggtgaagctggtggagtc) and JH primer (5′-tgcagagacagtgaccagag), and the cDNA sequence of the light chain was amplified by PCR using Vκ primer (5′-gacattgtgatgacacagtc) and Jκ primer (5′-tttcagctccagcttggtcc). The thus-obtained fragments were ligated with a linker and inserted into a vector, produced by New England Biolabs Inc., to transform Escherichia coli HB101. 96 clones were picked up from the thus-obtained colony and cultured. The clones were harvested after 16 hours of culturing.

Transgenic strains of these 96 clones were subjected to screening as described below. A 96-well plate was coated with recombinant influenza H1N1 HA (A/Puerto Rico/8/1934) at a concentration of 1 μg/mL at 4° C. overnight. After washing with PBS, Blocking One manufactured by Nacalai Tesque, Inc. was added to the plate at 100 μL/well to perform blocking at room temperature for 60 minutes. Subsequently, after washing with PBS, extracts of each clone were added to the well plates, and left to stand at 37° C. for 60 minutes for reaction. After washing with PBS, HRP conjugated anti MBP (×2000) produced by New England Biolabs Inc. was left to stand at 37° C. for 60 minutes for reaction. After washing with PBS, a coloring reagent manufactured by R&D Systems Science was reacted, and then H2SO4 was added to terminate the reaction. The absorbance was measured using a plate reader. A clone with the highest absorbance was used as anti-HA/PR8 in the following experiment.

A plasmid was extracted from anti-HA/PR8 clone using an Endofree Maxiprep Kit manufactured by QIAGEN. A preprotrypsin (PPT) leader sequence was inserted into the upstream of a maltose-binding protein of the above-obtained plasmid, thereby transforming Escherichia coli HB101. After culturing, the plasmid was collected, and the construction of the plasmid was confirmed by restriction enzyme treatment. A sense primer located upstream of the PPT and an antisense primer located downstream of the antibody gene were used to amplify the secretion signal sequence, maltose-binding protein gene sequence, and antibody gene sequence by PCR using an enzyme (KODplusNeo) manufactured by Toyobo, Co. Ltd. The PCR products were inserted into a retroviral vector plasmid (pMXspuro) to construct an anti-HA/PR8 retroviral vector plasmid.

A retrovirus was prepared in the following manner from the above-described anti-HA/PR8 retroviral vector plasmid.

Plat-GP packaging cells produced by Cell Biolabs, Inc. were co-transfected with an anti HA/PR8 retroviral vector plasmid and pCMV.VSV using Fugene 6 manufactured by Roche Ltd. Two days after transfection, the culture supernatant was collected, supplemented with polybrene (final concentration: 4 μg/mL), and used in the following infection experiment.

Human iPS cells (2,000/well) were suspension-cultured in the presence of mouse recombinant TGFβ and human recombinant BMP2 using a lipidure-coat plate (A-U96) manufactured by NOF Corporation, thereby forming embryoid bodies. Subsequently, the embryoid bodies were maintained in adherent culture in the presence of human recombinant BMP2, ascorbic acid, and insulin for 15 days, thereby preparing cartilage precursor cells.

The thus-obtained anti-HA/PR8-expressing retroviral vector was used to infect cartilage precursor cells, and then the cells were cultured for 2 days. On day 1 of culturing, the cells were divided into a group irradiated with 20 Gy of soft X-rays and a non-irradiated group, and 24 hours later, the culture supernatant was collected.

Anti HA/PR8 antibodies in the culture supernatant were measured by the following manner.

A 96-well plate was coated with recombinant influenza H1N1 HA(PR8) at a concentration of 1 μg/mL at 4° C. overnight. After washing with PBS, Blocking One manufactured by Nacalai Tesque, Inc. was added to the plate at 100 μL/well to perform blocking at room temperature for 60 minutes. Subsequently, after washing with PBS, the collected culture supernatant was added to the well plate and left to stand at 37° C. for 60 minutes for reaction. After washing with PBS, HRP conjugated anti MBP (×2000) produced by New England Biolabs Inc. was left to stand at 37° C. for 60 minutes for reaction. After washing with PBS, a coloring reagent manufactured by R&D Systems Science was reacted, and then H2SO4 was added to terminate the reaction. The absorbance was measured using a plate reader. FIG. 46 shows the results.

### Example 32

Mouse iPS cells were suspension-cultured in the absence of LIF, using a lipidure-coat plate manufactured by NOF Corporation, thereby forming embryoid bodies. Subsequently, the embryoid bodies were maintained in adherent culture in the presence of retinoic acid for 10 days to induce myoblast precursor cells. After infection with a GFP-expressing retroviral vector prepared using a Platinum Retroviral Expression System, the myoblast precursor cells were cultured for 2 days to induce differentiation of myoblasts. GFP expression in myoblasts was confirmed under a fluorescence microscope. This shows that in addition to chondrocytes, somatic cells that were induced to differentiate from iPS cells are also usable in the present invention.

### Example 33

Human iPS cells were suspension-cultured in the absence of LIF, using a lipidure-coat plate (A-U96) manufactured by NOF Corporation, thereby forming embryoid bodies. Subsequently, the embryoid bodies were maintained in adherent culture in the presence of retinoic acid for 10 days to induce myoblast precursor cells. After infection with a GFP-expressing retroviral vector prepared using a Platinum Retroviral Expression System, the myoblast precursor cells were cultured for 2 days to be induced to differentiate into myoblasts. GFP expression in myoblasts was confirmed under a fluorescence microscope. This shows that in addition to chondrocytes, somatic cells that were induced to differentiate from iPS cells are also usable in the present invention.

### Example 34

Human iPS cells (2,000/well) were suspension-cultured in the presence of mouse recombinant TGFβ and human recombinant BMP2 using a lipidure-coat plate (A-U96) manufactured by NOF Corporation, thereby forming embryoid bodies. Subsequently, the embryoid bodies were maintained in adherent culture in the presence of human recombinant BMP2, ascorbic acid, and insulin for 15 days, thereby preparing cartilage precursor cells. The cartilage precursor cells were infected with a secreted luciferase (MetLuc2) or mIL-12-expressing retroviral vector prepared using a Platinum Retroviral Expression System and cultured thereafter for 5 days. On day 5 of culturing, the cells were irradiated with 20 Gy of soft X-rays. Human iPS cell-derived chondrocytes (5×10⁶) were subcutaneously transplanted into immune-deficient mice (SCID mice). On day 90 after transplantation, the presence of tumor formation was examined. FIG. 47 shows the results.

1-15. (canceled)

16. A method for producing a grafting material comprising:
introducing a secreted protein gene into iPS cells and differentiating the iPS cells to obtain a grafting material expressing the secreted protein,
wherein the secreted protein gene is introduced during differentiating the iPS cells.

17. A method for producing a grafting material comprising:
introducing a secreted protein gene into iPS cells and differentiating the iPS cells to obtain a grafting material expressing the secreted protein,
wherein the method comprises exposing the grafting material to radiation and thereby eliminating the cell proliferation capability.

18. The method for producing a grafting material according to claim 16, wherein the grafting material contains chondrocytes.

19. The method according to claim 16, wherein the cells obtained by differentiating iPS cells form a cell population or cell mass, which can be transplanted or extracted as one cell population or cell mass.

20. The method according to claim 16, wherein the grafting material contains somatic cells (dedifferentiated cells) obtained by dedifferentiating somatic cells, inducing differentiation to other somatic cells after or during the dedifferentiation, and introducing the gene into the somatic cells thereduring.

21. A grafting material comprising iPS cell-derived differentiated cells, the grafting material obtained by the method according to any one of claims 16 to 20 and containing a secreted protein gene in such a manner that the secreted protein gene can be expressed.

22. The grafting material according to claim 21, wherein the differentiated cell is a chondrocyte.

23. The grafting material according to claim 21, wherein the grafting material is a population or mass of the differentiated cells.

24. The grafting material according to claim 21, which contains somatic cells (dedifferentiated cells) obtained by dedifferentiating the somatic cells, inducing differentiation to other somatic cells after or during the dedifferentiation, and introducing the gene into the somatic cells thereduring.

25. An agent for treating a disease caused by a deficiency, shortage, or hypofunction of a secreted protein, the agent comprising the grafting material obtained by any one of the methods of claims 16 to 20.

26. An agent for treating a disease caused by a deficiency, shortage, or hypofunction of a secreted protein, the agent comprising the grafting material obtained by any one of the grafting materials of claim 21 as an active ingredient.

27. The agent according to claim 25, wherein the secreted protein is at least one member selected from the group consisting of insulin, GLP-1, GLP-1(7-37) and like GLP-1 receptor agonist polypeptides, GLP-2, interleukins 1 to 33 (such as IL-1, IL-2, IL-3, IL-4, IL-5, IL-6, IL-7, IL-8, IL-9, IL-10, IL-11, IL-12, IL-13, IL-15, IL-17, IL-18, IL-21, IL-22, IL-27, IL-28, IL-33), interferons (α, β, γ), GM-CSF, G-CSF, M-CSF, SCF, FAS ligand, TRAIL, leptin, adiponectin, blood coagulation factor XIII/blood coagulation factor IX, lipoprotein lipase (LPL), lecithin cholesterol acyltransferase (LCAT), erythropoietin, apolipoprotein A-I, albumins, atrial natriuretic peptide (ANP), luteinizing hormone-releasing hormones (LHRH), angiostatin/endostatin, endogenous opioid peptides (enkephalins, endorphins and the like), calcitonin/bone morphogenetic proteins (BMP), pancreatic secretory trypsin inhibitors, catalase, superoxide dismutases, and antibodies.

28. The agent according to claim 26, wherein the secreted protein is at least one member selected from the group consisting of insulin, GLP-1, GLP-1(7-37) and like GLP-1 receptor agonist polypeptides, GLP-2, interleukins 1 to 33 (such as IL-1, IL-2, IL-3, IL-4, IL-5, IL-6, IL-7, IL-8, IL-9, IL-10, IL-11, IL-12, IL-13, IL-15, IL-17, IL-18, IL-21, IL-22, IL-27, IL-28, IL-33), interferons (α, β, γ), GM-CSF, G-CSF, M-CSF, SCF, FAS ligand, TRAIL, leptin, adiponectin, blood coagulation factor XIII/blood coagulation factor IX, lipoprotein lipase (LPL), lecithin cholesterol acyltransferase (LCAT), erythropoietin, apolipoprotein A-I, albumins, atrial natriuretic peptide (ANP), luteinizing hormone-releasing hormones (LHRH), angiostatin/endostatin, endogenous opioid peptides (enkephalins, endorphins and the like), calcitonin/bone morphogenetic proteins (BMP), pancreatic secretory trypsin inhibitors, catalase, superoxide dismutases, and antibodies.

29. The agent according to claim 25, wherein the disease is at least one member selected from the group consisting of diabetes, obesity, eating disorders, inflammatory bowel diseases, gastrointestinal disorders, vascular disorders, hemophilia, lipoprotein-lipase (LPL) deficiency, hypertriglyceridemia, lecithin cholesterol acyltransferase (LCAT) deficiency, hypoglobulia, low HDL cholesterol, hypoproteinemia, hypertension, heart failure, malignant melanoma, renal cancer, breast cancer, prostatic cancer, cancer metastasis, pain, osteoporosis, malignant tumors, hepatitis, allergies, multiple sclerosis, psoriasis, autoimmune diseases, pancreatitis, ischemic heart diseases and like ischemia reperfusion disorders.

30. The agent according to claim 26, wherein the disease is at least one member selected from the group consisting of diabetes, obesity, eating disorders, inflammatory bowel diseases, gastrointestinal disorders, vascular disorders, hemophilia, lipoprotein-lipase (LPL) deficiency, hypertriglyceridemia, lecithin cholesterol acyltransferase (LCAT) deficiency, hypoglobulia, low HDL cholesterol, hypoproteinemia, hypertension, heart failure, malignant melanoma, renal cancer, breast cancer, prostatic cancer, cancer metastasis, pain, osteoporosis, malignant tumors, hepatitis, allergies, multiple sclerosis, psoriasis, autoimmune diseases, pancreatitis, ischemic heart diseases and like ischemia reperfusion disorders.

31. A method for treating a disease comprising: administering the agent of claim 25 to a patient suffering from any of the diseases of diabetes, obesity, eating disorders, inflammatory bowel diseases, gastrointestinal disorders, vascular disorders, hemophilia, lipoprotein-lipase (LPL) deficiency, hypertriglyceridemia, lecithin cholesterol acyltransferase (LCAT) deficiency, hypoglobulia, low HDL cholesterol, hypoproteinemia, hypertension, heart failure, malignant melanoma, renal cancer, breast cancer, prostatic cancer, cancer metastasis, pain, osteoporosis, malignant tumors, hepatitis, allergies, multiple sclerosis, psoriasis, autoimmune diseases, pancreatitis, ischemic heart diseases and like ischemia reperfusion disorders.

32. A method for treating a disease comprising: administering the agent of claim 26 to a patient suffering from any of the diseases of diabetes, obesity, eating disorders, inflammatory bowel diseases, gastrointestinal disorders, vascular disorders, hemophilia, lipoprotein-lipase (LPL) deficiency, hypertriglyceridemia, lecithin cholesterol acyltransferase (LCAT) deficiency, hypoglobulia, low HDL cholesterol, hypoproteinemia, hypertension, heart failure, malignant melanoma, renal cancer, breast cancer, prostatic cancer, cancer metastasis, pain, osteoporosis, malignant tumors, hepatitis, allergies, multiple sclerosis, psoriasis, autoimmune diseases, pancreatitis, ischemic heart diseases and like ischemia reperfusion disorders.

33. A bank of a grafting material obtained by any one of the methods of claims 16 to 20.

34. A bank of a grafting material obtained by the grafting materials of claims 21.

35. The bank according to claim 31, wherein the grafting material is a chondrocyte.

36. The bank according to claim 32, wherein the grafting material is a chondrocyte.


SEQUENCE LISTING (4 sequences)

SEQ ID NO: 1 - DNA, 20 bp, Artificial
  Primer for PCR
gaggtgaagctggtggagtc

SEQ ID NO: 2 - DNA, 20 bp, Artificial
  Primer for PCR
tgcagagacagtgaccagag

SEQ ID NO: 3 - DNA, 20 bp, Artificial
  Primer for PCR
gacattgtgatgacacagtc

SEQ ID NO: 4 - DNA, 20 bp, Artificial
  Primer for PCR
tttcagctccagcttggtcc